(19) United States
(12) Patent Application Publication
(10) Pub. No.: US 2007/0026439 A1
(43) Pub. Date: Feb. 1, 2007

(54) Fluid processing device and method

(75) Inventors: Konrad Faulstich, Salem-Neufrach (DE); Mark F. Oldham, Los Gatos, CA (US)

Correspondence Address: KILYK & BOWERSOX, P.L.L.C., 3603 CHAIN BRIDGE ROAD, SUITE E, FAIRFAX, VA, 22030, US

(73) Assignee: Applera Corporation, Foster City, CA (US)

(21) Appl. No.: 11/487,729

(22) Filed: Jul. 17, 2006

Related U.S. Application Data
(60) Provisional application No. 60/699,782, filed on Jul. 15, 2005.

(51) Int. Cl. C12Q 1/68; C12P 19/34; C12M 1/34

(52) U.S. Cl. 435/6; 435/287.2; 435/91.2

(57) ABSTRACT

A fluid processing device and methods are provided that can process one or many different fluid samples, detection for each of which can be multiplexed to detect the presence or absence of each of a panel of target sequences, for example 20 different target sequences. The device can comprise a substrate and one or more fluid processing pathways at least partially defined by the substrate. Each fluid processing pathway can comprise a pre-amplification region and two or more amplification regions disposed downstream from and in fluid communication with the pre-amplification region. A burstable valve can be disposed along each fluid processing pathway and the downstream regions can contain pre-loaded ammonia gas to draw an amplified sample downstream.

### CROSS REFERENCE TO RELATED APPLICATION

The present application claims a benefit from earlier filed U.S. Provisional Patent Application No. 60/699,782, filed Jul. 15, 2005, which is incorporated herein in its entirety by reference.

### INTRODUCTION

The present teachings relate to a device, a system, and methods, for processing fluids. More particularly, the present teachings relate to devices that manipulate, process, or otherwise alter fluid samples.

### SUMMARY

According to various embodiments, a fluid processing device is provided wherein a plurality of different nucleic acid sequences contained in a sample can be pre-amplified to produce a plurality of different pre-amplified sequences and one or more target nucleic acid sequences of the plurality of different pre-amplified sequences can then be amplified, using a single device. The fluid processing device can comprise a microfluidic device.

According to various embodiments, a fluid processing device is provided that can comprise: a substrate that can comprise a first surface, an opposing second surface, and a thickness; and one or more fluid processing pathways at least partially defined by the substrate, the one or more fluid processing pathways each can comprise a first region that can comprise pre-amplification reaction components disposed therein and can be adapted to pre-amplify a plurality of different nucleic acid sequences present in a sample to produce a plurality of pre-amplified sequences and two or more second regions each of which can be in fluid communication with the first region and can comprise amplification reaction components disposed therein that can be adapted to amplify one or more target sequences of the plurality of pre-amplified sequences to produce one or more amplified target sequences.

According to various embodiments, a fluid processing device is provided that can comprise: a substrate having a first surface and an opposing second surface; and one or more fluid processing pathways that can be at least partially defined by the substrate, the one or more fluid processing pathways each can comprise at least one heat-mediated, pressure-actuated valve that can be adapted to burst when a pressure of at least two atmospheres is exerted across the valve and the valve is heated to a temperature of from about 100° C. to about 150° C., for example, from about 110° C. to about 130° C.

According to some embodiments, a fluid processing device is provided that can comprise: a substrate having a first surface and an opposing second surface; and one or more fluid processing pathways that can be at least partially defined by the substrate, the one or more fluid processing pathways each can comprise a first region and one or more sealed regions disposed downstream from and in fluid communication with the first region, each of the one or more sealed regions can comprise ammonia gas. In some embodiments the one or more fluid processing pathways can further comprise a valve disposed between and in fluid communication with the first region and the one or more sealed regions.

According to some embodiments, a fluid processing system is provided that can comprise a fluid processing device, and a detector in optical and/or electrochemical communication with two or more second regions of each fluid processing pathway of the fluid processing device, the detector can be adapted to detect, in the two or more second regions, one or more amplified target sequences each of which can be labeled with a respective detectable label. The fluid processing system can comprise a thermal cycling device. If included, the thermal cycling device can comprise, for example, a peltier device or other known heating device. Exemplary peltier devices that can be used include those described in U.S. patent application Ser. No. 10/926,915 filed Aug. 26, 2004, which is incorporated herein in its entirety by reference. The thermal cycling device can provide two or more different temperature zones, for example, to heat two sections of the fluid processing device to two different temperatures and/or to provide a hot zone and a cool zone.

According to some embodiments, a fluid processing method is provided that comprise: providing a fluid processing device that can comprise one or more fluid processing pathways, each fluid processing pathway can comprise a first region in fluid communication with two or more second regions; introducing a fluid sample that can comprise a plurality of different nucleic acid sequences into the first region of the fluid processing device; pre-amplifying a plurality of different nucleic acid sequences in the first region to produce a pre-amplified fluid sample that can comprise a plurality of pre-amplified nucleic acid sequences; moving the pre-amplified fluid sample from the first region to the two or more second regions; and amplifying at least one respective target sequence of the plurality of pre-amplified nucleic acid sequences in each of the two or more second regions, to produce at least one respective amplified target sequence in each of the two more second regions.

According to some embodiments, a fluid processing method is provided that can comprise: providing a fluid processing device that can comprise one or more fluid processing pathways each of which can comprise a first region and at least one sealed region disposed downstream from and in fluid communication with the first region, wherein the at least one sealed region can comprise ammonia gas; retaining a fluid sample in the first region; and contacting the fluid sample with the ammonia gas, such that the fluid sample is drawn from the first region as the ammonia gas dissolves into the fluid sample. This fluid drawing through solubilization can occur more than once, for example, to sequentially draw a fluid and/or reaction product thereof into two or more different regions.

According to various embodiments, pre-amplification and amplification of one or more target nucleic acid sequences can be accomplished in a single fluid processing device, for example, a single microfluidic processing device. In some embodiments, pre-amplification and amplification can be accomplished in a single fluid processing device, along with one or more of sample preparation, sequencing reactions and detecting reactions.

According to some embodiments, a fluid processing device and methods are provided that can process up to 50 different fluid samples each multiplexed for a panel of pathogens, for example 20 pathogens. According to some embodiments, a fluid processing device and methods are provided that can identify pathogens, antibiotics resistance, origin of species, mutations, cancer, or other genomic disorders. The fluid processing device and methods can be sensitive to a single molecule and can be strain specific.

According to various embodiments, a method is provided that can comprise a multiplex amplification process, for example, a multiplex PCR process. The process can comprise pre-amplifying a large region encompassing more than one segment of a nucleic acid molecule using primers outside the target area in a first or pre-amplification region of a fluid processing pathway of a fluid processing device, that can be followed by amplification of each target area using specific primers for each site in a second or amplification region in fluid communication with the first or pre-amplification region of the device. This method allows for the simultaneous detection of more than one polymorphic region in a particular gene or several genes.

Additional features and advantages of the present teachings will be set forth in part in the description that follows, and in part will be apparent from the description, or may be learned by practice of the present teachings. The objectives and other advantages of the present teachings will be realized and attained by the means of the elements and combinations particularly pointed out in the description that follows.

It is to be understood that both the foregoing general description and the following detailed description are exemplary and explanatory only and are intended to provide a further explanation of the present teachings.

### DRAWINGS

Various embodiments of the present teachings are exemplified in the accompanying drawings. The teachings are not limited to the embodiments depicted in the drawings, and include similar structures and methods as set forth in the following description and as would be known to those of ordinary skill in the art in view of the present teachings. In the drawings:

FIG. 1 illustrates a plan view of a fluid processing device, according to various embodiments;

FIG. 2 illustrates a plan view of a fluid processing device, according to some embodiments;

FIG. 3 illustrates a cross-sectional view of the fluid processing device illustrated in FIG. 2;

FIG. 4 illustrates a plan view of a fluid processing device according to various embodiments;

FIGS. 5-10 illustrate a system according to various embodiments of the present teachings and comprising a pre-amplification array, a mixing array, and a microfluidics card; and

FIG. 11 depicts a system according to other various embodiments of the present teachings.

It is to be understood that the following descriptions are exemplary and explanatory only. The accompanying drawings are incorporated in and constitute a part of this application and illustrate several exemplary embodiments with the description. Reference will now be made to various embodiments, examples of which are illustrated in the accompanying drawings. Wherever possible, the same reference numbers are used in the drawings and the description to refer to the same or like parts.

### DESCRIPTION

### I. General

Throughout the application, descriptions of various embodiments use “comprising” language; however, it will be understood by one of skill in the art, that in some specific instances, an embodiment can alternatively be described using the language “consisting essentially of” or “consisting of.”

For purposes of better understanding the present teachings and in no way limiting the scope of the teachings, it will be clear to one of skill in the art that the use of the singular includes the plural unless specifically stated otherwise. Therefore, the terms “a,” “an” and “at least one” are used interchangeably in this application.

Unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. In some instances, “about” can be understood to mean a given value ±5%. Therefore, for example, about 100 nl, could mean 95-105 nl. At the very least, each numerical parameter should at least be construed in light of the number of reported significant digits and by applying ordinary rounding techniques.

As used herein, the term “plurality” can be understood as “two or more.” Herein, the term “two or more” is used synonymously with the term “plurality.”

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Herein, the term nucleic acid sequence refers to any sequence of nucleotide bases, for example, a sequence held together by a sugar-phosphate backbone. The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2 ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O6-methylguanine, N6-methyladenine, O4-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-anomeric nucleotides, 1′-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). Exemplary LNA sugar analogs within a polynucleotide include, but are not limited to, the structures: 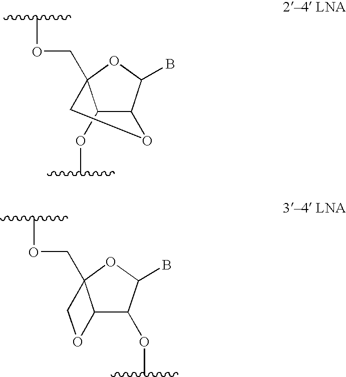
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N9-position of the nucleotide base. When the nucleotide base is pyrimidine, e.g. C, T or U, the pentose sugar is attached to the N1-position of the nucleotide base, except for pseudouridines, in which the pentose sugar is attached to the C5 position of the uracil nucleotide base (see, e.g., Kornberg and Baker, (1992) DNA Replication, 2nd Ed., Freeman, San Francisco, Calif.).

One or more of the pentose carbons of a nucleotide may be substituted with a phosphate ester having the formula: 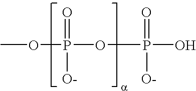
where α is an integer from 0 to 4.

In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g.—thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog,” as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of nucleotide “analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone. Also included are intercalating nucleic acids (INAs, as described in Christensen and Pedersen, 2002), and AEGIS bases (Eragen, U.S. Pat. No. 5,432,272).

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H+, NH4+, trialkylammonium, Mg2+, Na+ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 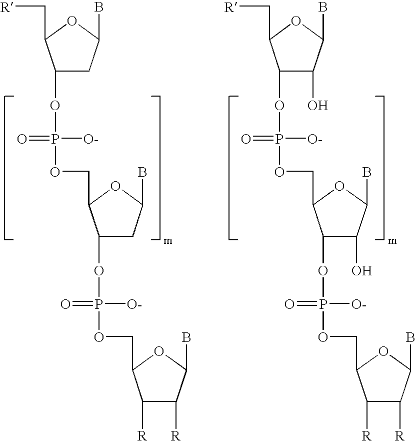
wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or 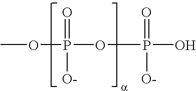
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C1C4 alkylphosphonate, e.g. methylphosphonate; (ii) phosphoramidate; (iii) C1C6 alkyl-phosphotriester; (iv) phosphorothioate; and (v) phosphorodithioate.

According to various embodiments, a target nucleic acid sequence can comprise any nucleic acid sequence of interest, for example, a nucleic acid, a SNP, a nucleic acid containing all or a portion of a polymorphic region of a gene of interest, or the like.

According to various embodiments, methods are provided that refer to processes or actions involved in sample preparation and analysis. It will be understood that in various embodiments a method can be performed in the order of processes as presented, however, in related embodiments the order can be altered as deemed appropriate by one of skill in the art in order to accomplish a desired objective.

According to various embodiments, a fluid sample can comprise an aqueous or a non-aqueous sample. An aqueous or a non-aqueous sample can comprise any nucleic acid containing material, for example, a biological material. A nucleic acid containing material can comprise, for example, one or more of: blood; a cell sample; a sub-cellular organelle, for example, mitochondria or chloroplasts; a cell lysate; a tissue sample, for example, skin; cell culture medium; a body fluid, for example, saliva, urine, or effusion; biopsy medium; and the like. Such nucleic acid containing material can comprise any source, for example, a human source, an animal source, a plant source, a bacterial source, a viral source, or the like. A nucleic acid containing sample can be processed to obtain nucleic acid prior to or simultaneously with, pre-amplification. Nucleotides and nucleic acids can be obtained prior to pre-amplification, using methods known to those of skill in the art.

According to various embodiments, processing or reaction components can comprise one or more components necessary or desirable for use in one or more processes, for example, components that in any way affects how a desired reaction can proceed. A processing component can comprise a reactive component. However, it is not necessary that the component participate in the reaction. The processing component can comprise a non-reactive component. The processing component can comprise a recoverable component that can comprise, for example, a solvent and/or a catalyst. The processing component can comprise a promoter, accelerant, or retardant that is not necessary for a reaction but affects the reaction, for example, affects the rate of the reaction. The term “reaction component” is used synonymous with the term “processing component,” as herein described. Suitable processes can comprise one or more of a sample preparation process, a sample purification process, a pre-amplification process, a pre-amplified product purification process, an amplification process, an amplified product purification process, a separation process, a sequencing process, a sequencing product purification process, a labeling process, a detecting process, or the like. Processing components can comprise sample preparation components, purification components, pre-amplification reaction components, amplification reaction components, sequencing reaction components, or the like. The skilled artisan can readily select and employ suitable components for a desired reaction or process, without undue experimentation.

According to some embodiments, processing or reaction components can be disposed in one or more regions or channels using any methods known in the art. For example, components can be sprayed and dried, delivered using a diluent, injected using a capillary, a pipette, and/or a robotic pipette, or otherwise disposed in the regions or channels.

According to some embodiments, a fluid processing device is provided that can comprise one or more fluid processing pathways that can each comprise one or more elements, for example, one or more of a region, a channel, a branch channel, a valve, a flow splitter, a vent, a port, an access area, a via, a bead, a reagent-containing bead, a cover layer, a reaction component, a flow combiner, a flow merger, intersecting flow pathways, any combination thereof, and the like. Any element can be in fluid communication with another element, wherein “fluid communication” can be either understood as being in direct fluid communication, for example, two regions can be in fluid communication with each other via an unobstructed channel connecting the two regions, or be understood as being adapted to be in fluid communication, for example, two regions can be adapted to be in fluid communication with each other when they are connected via a channel or other passageway that comprises a closed valve disposed therein, wherein fluid communication can be established between the two regions upon opening the valve in a channel. As used herein, the term “in fluid communication” refers to in direct fluid communication and/or adapted to be in direct fluid communication, unless otherwise expressly stated. The term “in valved fluid communication” is also used herein and refers to elements wherein a valve is disposed between the elements, such that upon opening or actuating the valve, fluid communication between the elements can be established.

According to various embodiments, the valves that can be used as described herein can comprise, for example, dissolvable valves, swellable valves, and/or composite valves, for example, as described in U.S. patent application Ser. No. 11/252,821, filed Oct. 18, 2005, Ser. No. 11/252,912, filed Oct. 18, 2005, Ser. No. 11/252,915, filed Oct. 18, 2005, and Ser. No. 11/252,914, filed Oct. 18, 2005, all of which are incorporated herein in their entireties by reference. According to various embodiments, valving can comprise sealing a channel with oil as described, for example, in U.S. patent application Ser. No. 11/380,327, which is incorporated herein in its entirety by reference.

Reactions involving nucleic acids can comprise enzymatic reactions in which a nucleic acid is amplified to increase the amount of target nucleic acid for analysis. Other reactions can comprise, for example, primer extension reactions, sequencing reactions, fragmentation reactions (e.g., using specific endonucleases), cleavage reactions of mismatched heteroduplexes of nucleic acids, oligonucleotide ligation reactions and single-stranded conformation reactions. According to various embodiments, the second or downstream amplification reaction can be a product amplification reaction as opposed to a target sequence amplification reaction. As such, the target sequence is not amplified but instead one or more reaction products thereof are produced in a multi-fold manner to eventually form a multi-fold detectable product. An exemplary detection assay of this variety is the Invader detection assay available from Third Wave Technologies of Madison, Wis. Exemplary detection assays are described in U.S. Pat. No. 6,706,471 to Brow et al., and in U.S. Patent Application Publication No. US 2004/0014067, published Jan. 22, 2004, which are incorporated herein in their entireties by reference.

Nucleic acid amplification reactions can comprise polymerase chain reaction (PCR) that can be performed according to any methods known in the art. For example, in one PCR protocol, genomic DNA of a cell is exposed to two PCR primers and amplification is performed for a number of cycles sufficient to produce a required amount of amplified DNA. The primers can be located, for example, between about 50 and 350, between about 50 and 500, up to about 1000 or more base pairs apart, up to about 10,000 base pairs apart, or up to 100,000 or more base pairs apart.

If a multiplex PCR amplification is to be carried out, initially a large region encompassing more than one segment of a nucleic acid molecule can be amplified using primers outside the area, followed by amplification of each sub-region or segment using specific primers for each site, for example, nested PCR. Some of the limitations of multiplex PCR include partial binding between PCR primers or between PCR primers and other primers or other regions of the genomic DNA apart from the target site, thus resulting in side products and reduced yields of the desired PCR products. Those of ordinary skill in the art are familiar with the design and limitations of multiplex PCR.

Exemplary multiplex methods and apparatus that can be used in conjunction with the present teachings include those described, for example, in U.S. Patent Application Publication No. US2004/0175733, published Sep. 9, 2004, which is incorporated herein in its entirety by reference.

Additional methods of amplifying nucleic acids can comprise, but are not limited to, mini-PCR, ligase chain reaction (LCR) [Wiedmann et al. (1994) PCR Methods Appl. Vol. 3, Pp. 57-64; Bamay (1991) Proc. Natl. Acad. Sci USA 88:189-93], strand displacement amplification (SDA) [Walker et al. (1994) Nucleic Acids Res. 22:2670-77], RT-PCR [Higuchi et al. (1993) Bio/Technology 11:1026-1030], rolling circle amplification, Recombinase Polymerase Amplification (Armes et al., WO03072805; Piepenburg et al., US2005112631), EXPAR (van Ness et al., WO2004067726), Isothermal Nucleic Acid Amplification resulting in a spatially localized product (Saba, http://wbabin.net/saba/saba17.htm), autocatalytic methods, such as those using QJ replicase, TAS, 3SR, and any other suitable method known to those of skill in the art.

Alternative amplification methods can comprise self sustained sequence replication (Guatelli et al. (1990) Proc. Natl. Acad. Sci. U.S.A. 87:1874-1878), transcriptional amplification system (Kwoh et al. (1989) Proc. Natl. Acad. Sci. U.S.A. 86:1173-1177), Q-Beta Replicase (Lizardi et al. (1988) Bio/Technology 6:1197), or any other nucleic acid amplification method, followed by the detection of the amplified molecules using techniques well known to those of skill in the art and disclosed herein. These detection schemes are especially useful for the detection of nucleic acid molecules if such molecules are present in very low numbers. Alternatively, allele specific amplification technology, which depends on selective PCR amplification may be used. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). In addition it may be desirable to introduce a restriction site in the region of a mutation to create cleavage-based detection (Gasparini et al. (1992) Mol. Cell Probes 6:1).

Primer Extension Reactions: primer extension reactions involve the specific termination of polymerase-mediated nucleic acid chain elongation by the incorporation of a chain terminator, e.g., a dideoxynucleotide, into the elongation reaction. Several primer extension-based methods are known in the art and have been used for determining the identity of a particular nucleotide in a nucleic acid sequence. In general, a primer is prepared that specifically hybridizes adjacent to a site of interest, e.g., a polymorphic site, in a particular nucleic acid molecule. The primer is then extended in the presence of one or more dideoxynucleotides, typically with at least one of the dideoxynucleotides being the complement of the nucleotide that is polymorphic at the site.

Fragmentation Reactions: The presence or absence of one or more mutations, such as polymorphisms, within specific nucleic acids, including PCR products or other amplification products, can be determined from fragments of these nucleic acids. The fragments can be generated by different chemical and/or enzymatic reactions. For any of these fragmentation reactions, the molecular weights of the nucleic acid fragment(s) obtained after the reaction can be determined by, for example, by electrophoresis, capillary electrophoresis, mass spectrometry, or the like.

The target nucleic acid can be characterized through its whole fragmentation pattern, characterizing the complete sequence or through selected parts of the fragmentation pattern. Certain fragments of the nucleic acid can be selectively isolated and purified, for example through capture by hybridization on the substrate or through capture on beads; or through other specific interactions, like Biotin/Streptavidin affinity of one or more fragments. Methods for isolating and purifying all or most of the generated fragments, includes for example specific and non-specific (e.g., through the use of polyinosine) capture by hybridization on a substrate as well as substrates that bind fragments through ionic, hydrogen bond or hydrophobic interaction, chelating ligands, affinity interaction or through other means known to those skilled in the art.

One method for generating fragments of nucleic acids, preferably from amplification products, is the use of one or more restriction enzymes. Analyzing the number, size and/or composition of the product(s) of the reaction will provide information about the nucleic acid and its variants at one or multiple sites. For example, a specific nucleotide polymorphism within Sequencing Reactions: a variety of nucleic acid sequencing reactions are known in the art and can be used to identify a particular nucleic acid. Exemplary sequencing reactions include those based on techniques developed by Maxam and Gilbert [(1977) Proc. Natl. Acad. Sci. USA 74:560] or Sanger [Sanger et al. (1977) Proc. Natl. Acad. Sci. U.S.A. 74:5463]. It will be evident to one skilled in the art that, for certain embodiments, the occurrence of only one, two or three of the nucleic acid bases need be determined in the sequencing reaction. For instance, A-track sequencing or an equivalent, e.g., where only one nucleotide is detected, can be carried out. Other sequencing methods are known (see, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”).

Multiplex reactions can be carried out in one or more of the first region and the second regions. In an exemplary embodiment, a 100-plex reaction can be carried out in a first region, for example, whereby 100 different target nucleic acid sequences are amplified. The 100-plex product can then be communicated to a plurality of second regions, for example, to 25 reaction regions. The communicating can comprise the formation of a fluid communication from the first region to each of the second regions, for example, using a plurality of channels and first splitters, and/or opening a valve. In the 25 second regions, another multiplex reaction can be carried out, for example, a different 4-plex reaction in each of the 25 second regions. In another embodiment, a 20-plex reaction can be carried out in first region followed five different 4-plex reactions in five respective second regions.

In yet another embodiment, an initial cDNA sample can be divided into 24 different first regions. In each of the 24 first regions, a different respective 1280-plex reaction can be carried out. The amplified product in each first region can then be moved into 256 respective second regions wherein a different respective five-plex amplification and/or detection can occur.

According to some embodiments, multiplex reactions can be carried out and used for the detection of single nucleotide polymorphisms (SNP's). In some embodiments, reaction products can be ligated onto molecular rate modifiers and then be resolved or detected on a capillary electrophoretic analyzer. According to some embodiments, single nucleotide polymorphisms can be detected in the second regions. In some embodiments SNP-plex reactions can be carried out in the first and/or second regions.

Oligonucleotide Ligation Reaction: in another nucleic acid reaction scheme, referred to as oligonucleotide ligation, two oligonucleotides, designed to be capable of hybridizing abutting to sequences of a single strand of a target nucleic acid, are mixed with sample nucleic acid. If the precise complementary sequence is found in a sample nucleic acid, the oligonucleotides will hybridize such that their termini abut, and create a ligation substrate. Thus, a nucleic acid in a sample can be detected using an oligonucleotide ligation assay (OLA), as described, e.g., in Landegren et al., Science 241:1077-1080 (1988). Nickerson et al. have described a nucleic acid detection assay that combines attributes of PCR and OLA [Nickerson et al. (1990) Proc. Natl. Acad. Sci. U.S.A. 87:8923-89271. In this method, PCR is used to achieve the exponential amplification of target DNA, which is then detected using OLA. In an alternative embodiment OLA can be used first, followed by PCR, for example, highly multiplexed PCR. All the above references are incorporated herein in their entireties by reference.

Several techniques based on this OLA method have been developed and can be used to detect specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoramidate linkage. Other techniques that can be used include OLA-PCR techniques and PCR-OLA techniques.

In other protocols, based on the ligase chain reaction (LCR), a target nucleic acid is hybridized with a set of ligation educts and a thermostable DNA ligase, so that the ligase educts become covalently linked to each other, forming a ligation product. Reagents that can be used in Nucleic Acid Reactions: as is evident from the types of reactions involving nucleic acids, a number of reagents can be used in such reactions. Reagents include enzymes (e.g., polymerases, endonucleases, exonucleases, S1 nuclease, ligases), primers, oligonucleotides, deoxynucleoside triphosphates (dNTPs) and dideoxynucleoside triphosphates (ddNTPs).

In other protocols, nucleic acid sequences can be attached to proteins, Antibodies and Antigens. Those methods are referred to in the literature as Immuno-PCR (Sano et al., Science 258, 120-122 (1992), Sims et al., Anal. Biochem 281, 230-232 (2000)) and Proximity Ligation Assay (Fredriksson et al., Nature Biotechnology 20, 473-477 (2002)). They are more sensitive than ELISA methods, which are traditionally used for protein detection. To use those methods in combination with the here described devices and methods extends the usefulness to protein detection and can facilitate highly multiplexed protein detection.

Primers: primers refer to nucleic acids which are capable of specifically hybridizing to a nucleic acid sequence (often referred to as a template) at a position which is adjacent to a region of interest, for example, a polymorphic region. A primer can be extended through the action of an enzyme, e.g., a polymerase, in a process whereby nucleotides or analogs thereof that are complementary to the template adjacent to the primer are added to the growing nucleotide chain. For example, if an RNA template is used, an oligodeoxynucleotide primer can be extended through the action of reverse transcriptase to generate a cDNA complementary to the RNA template. If a DNA template is used, a primer can be extended through the action of a DNA polymerase.

A primer can be used alone, for example in a primer extension reaction designed to provide information on the identity and/or presence of a target nucleic acid, or a primer can be used together with at least one other primer or probe, e.g., in an amplification reaction. For amplifying at least a portion of a nucleic acid, a forward primer (i.e., 5′ primer) and a reverse primer (i.e., 3′ primer) will preferably be used. Forward and reverse primers hybridize to complementary stands of a double-stranded nucleic acid, such that upon extension from each primer, a double stranded nucleic acid is amplified.

Primers (RNA, DNA (single-stranded or double-stranded), PNA and their analogs) described herein may be modified without changing the substance of their purpose by terminal addition of nucleotides designed, for example, to incorporate restriction sites or other useful sequences.

A primer can be prepared according to methods well known in the art and described, e.g., in Sambrook, J. and Russell, D. (2001) Molecular Cloning: A Laboratory Manual, Third Ed., Cold Spring Harbor Laboratory Press, Cold Spring Harbor, N.Y. For example, discrete fragments of the DNA can be prepared and cloned using restriction enzymes. Alternatively, primers can be prepared using the Polymerase Chain Reaction (PCR) using primers having an appropriate sequence or they can be synthesized.

Primers, and in particular primers used in reactions conducted in methods of detecting allelic variants, are of sufficient length to specifically hybridize to portions of an allele at polymorphic sites. Typically such lengths depend upon the complexity of the source organism genome. For humans such lengths are at least 14-16 nucleotides, and typically may be 20, 30, 50, 100 or more nucleotides.

Nucleosides/Nucleotides: many reactions involving nucleic acids include deoxynucleoside triphosphates (dNTPS) and dideoxynucleoside triphosphates (ddNTPs) as the building blocks which can be used, for example, to extend a primer in extension, amplification and sequencing reactions. In certain reactions, it can be desirable to use modified dNTPS to facilitate identification and/or detection of the products of the reactions or to distinguish the products of different reactions. For example, a molecular weight difference between the nucleic acid products of different reactions can be achieved either by the nucleic acid sequence itself (composition or length) or by the introduction of mass-modifying functionalities into the products. For example, mass modifications can be incorporated during a nucleic acid amplification process.

Types of Oligonucleotides: the sequence, length and composition of an oligonucleotide will vary depending upon the nature of the nucleic acid to be captured. The oligonucleotide can be specific for each assay product or can be complementary to a common region of two or more allelic variants of a polymorphic site. For example, in a primer extension reaction assay, the surface immobilized complement oligonucleotide can hybridize to the extension product that results from both alleles of the polymorphic site. This is because the hybrid does not form with the polymorphic region. The oligonucleotide hybridizes with the extension product 5′ to the polymorphic region. But, each oligonucleotide only hybridizes with alleles of a single polymorphic region.

According to various embodiments, a generic oligonucleotide (“zip code” oligonucleotide) can be immobilized on the substrate. A zip code oligonucleotide can be any length and is typically 6 to 25 nucleotides in length. The captured assay product has a zip code complement sequence to allow for hybridization to the surface-bound oligonucleotide.

According to various embodiments, zip codes can be useful in non-immobilization techniques, for example, zip codes can be used as primers for a secondary amplification reaction. Various methods using universal PCR and/or zip codes, that can be carried out according to various embodiments can comprise the methods and use of the components described, for example, in U.S. patent application Ser. No. 11/090,830 to Andersen et al., and Ser. No. 11/090,468 to Lao et al., both filed Mar. 24, 2005, in U.S. Pat. No. 6,605,451 to Marmaro et al., and in international patent application publication no. WO 2004/051218 to Andersen et al., which are incorporated herein in their entireties by reference. Real time PCR and resequencing are two exemplary methods that can be carried out using such methods and devices.

Zip codes can be shared by assay products used to capture and detect different polymorphisms in one location. Different sets of zip codes and complement zip code sequences can be used to separate assay products of different polymorphic sites in different locations, as single assay products as well as in small groups of different assay products. The use of generic zip code sequences simplifies manufacturing and quality control of the substrate. The described strategies facilitate the processing and analysis of multiplexed samples.

A modification of the zip code approach is to incorporate a cleavable site in the extended primer. For example, the zip code sequence can be cleaved from the assay product to create assay products that are more suitable for the method of analysis, like mass spectrometry. The cleavable site can be an enzymatic or base-cleavable site. For example, a single ribonucleotide in a sequence of deoxyribonucleotides is cleavable by ribonucleases or by base. An abasic site can be incorporated during the synthesis of the oligonucleotide or induced by enzymes and chemicals and cleaved under basic conditions or with enzymes. When MALDI-TOF mass spectrometry is used for analysis of reaction products, enzymes or reagents for cleavage can be added to the captured nucleic acid along with matrix. Other alternatives include acid-cleavable sites (e.g., sites that can be cleaved by matrix for mass spectrometry or matrix additives) as in the case of phosphoramidate bonds [see, e.g., Shchepinov et al. (2001) Nucleic Acids Res. 29:3864-3872] or photocleavable sites, such as may be cleaved by a laser in laser-based mass spectrometry. Disulfide bonds can also be used and cleaved in the presence of a reducing agent such as dithiothreitol.

In another embodiment, the surface bound oligonucleotide is the amplification product which becomes attached to an activated substrate or chip. The substrates are activated up to the point of oligonucleotide addition as described herein or in Example 2. Attachment of the PCR product to the surface occurs during and/or after the PCR. Chemical attachment of the PCR product is achieved through a 5′-modification of the PCR primer(s). Also, passive attachment of the PCR product to the surface can occur via for example, electrostatic interactions, Van der Waals forces and hydrogen bonds. The assay product, e.g., primer extension product, is captured by hybridization to the surface immobilized amplification product.

Alternatively, as previously described, a generic oligonucleotide (“zip code” oligonucleotide) can be immobilized on the substrate. The amplification product has an attached zip code complement sequence to allow for hybridization to the bound oligonucleotide. The amplification product simultaneously captures the assay product. The zip code oligonucleotide can be modified, in a way that the stability of the formed hybrid can be significantly increased, for example, by using RNAs, LNAs, (PNAs) or other modified nucleic acid derivatives, completely or partly within the sequence. Zip code and the corresponding region of the amplification product can as well be permanently crosslinked with each other through reactive groups in the formed hybrid. In another embodiment, a generic zip code oligonucleotide is immobilized on the substrate. One strand of the amplification product is designed to have a single-stranded overhang sequence on one end. After PCR or any other method used for amplification, capture by hybridization is mediated by a third oligonucleotide with a sequence that is to one part complementary to the zip code on the surface, to the other part complementary to the additional overhang sequence on the target strand of the amplification product. Thus mediating the contact between amplification product and zip code sequence on the surface, the formed hybrid can furthermore be used to permanently link the target strand to the surface, for example by using the ligase reaction. The covalent attachment permits the isolation of a single-stranded amplification product by washing the second strand and the mediating oligonucleotide away under suitable buffer conditions. The single strand isolation on the substrate can for example be followed by reactions to identify SNP sites within the immobilized target DNA by primer extension reactions. The assay products are finally captured and conditioned for analysis through hybridizing with the immobilized target DNA.

Multiplexing: multiplex methods allow for the simultaneous detection of more than one polymorphic region in a particular gene or several genes. Multiplexing at respective locations can be achieved by utilizing a different capture oligonucleotide for the product of each set of specific assay reactions. According to some embodiments, different capture oligonucleotides can comprise immobilized capture oligonucleotides. According to some embodiments, different capture oligonucleotides can comprise non-immobilized capture oligonucleotides and different sets of: probes; and at least one of excitation sources and detectors. Alternatively, rather than using immobilized capture oligonucleotides, localized primers can instead be used, optionally with probes. Exemplary systems that can be used for multiplexing include detection systems that differentiate fluorescent signals based on excitation wavelength, based on emission wavelength, based on both excitation and emission wavelengths, or based on other analytical techniques. An exemplary system is described in U.S. patent application Ser. No. 10/440,852, filed May 19, 2003, which is incorporated herein in its entirety by reference.

### II. Device and System

According to some embodiments a fluid processing device is provided. The device can comprise a substrate that can comprise, for example, a top or a first surface, and one or more fluid processing pathways that can be provided in communication with and/or can be defined by, for example, at least a portion of the top or first surface of the substrate. Two or more of the one or more fluid processing pathways can be provided substantially parallel to each other. The one or more fluid processing pathways can be provided, for example, in a top or first surface of a substrate, on a top or first surface of a substrate, in a substrate, in a bottom or second surface of a substrate, on a bottom or second surface of a substrate, in an edge of a substrate, on an edge of a substrate, or any combination thereof. The one or more fluid processing pathways can comprise one or more of, for example, a region, a plurality of regions, a channel, a valve, a flow splitter, a branch channel, an access area, a port, intersecting channels, flow combiners, flow diverters, intersecting flow pathways, and a combination thereof. A region can comprise any area that can be used to, for example, retain, process, react, store, incubate, transfer, purify, or the like, a fluid sample. Two or more fluid processing pathways can be provided substantially parallel to each other whereby second regions of two or more substantially parallel fluid processing pathways can be aligned and can define an axis substantially perpendicular to an axis defined by the two or more substantially parallel fluid processing pathways. A fluid processing device can comprise different levels and layers of fluid processing pathways that can comprise, for example, different levels and layers of channels and regions. For example, a tiered, multi-channel device can comprise one or more fluid processing pathways that traverse different heights or levels in the substrate.

According to various embodiments, the fluid processing device can comprise one or more pathways that each comprises a first reaction region, a second reaction region, and one or more additional regions. The one or more additional regions can comprise, for example, one or more purification regions, flow splitter regions, product collection regions, reactant loading regions, combinations thereof, and the like, for example, as described in U.S. patent application Ser. No. 10/336,274, filed Jan. 3, 2003, and Ser. No. 10/628,781, filed Jul. 28, 2003, both of which are incorporated herein in their entireties by reference. The various regions can be separated by valves and/or by oil, as described herein.

According to various embodiments, a fluid processing device is provided that can comprise a substrate. The substrate can comprise an insoluble support. The substrate can comprise any insoluble or solid material, for example, silicon, silica gel, glass (e.g. controlled-pore glass (CPG)), nylon, Wang resin, Merrifield resin, Sephadex®, Sepharose®, cellulose, a metal surface (e.g., steel, gold, silver, aluminum, and copper), a plastic material (e.g., polyethylene, polypropylene, polyamide, polyester, polyvinylidenedifluoride (PVDF), polydimethylsiloxane, and RTV's. The substrate can comprise, for example, one or more of flat supports that can comprise one or more of glass fiber filters, silicon surfaces, glass surfaces, metal surfaces (steel, gold, silver, aluminum, and copper), and plastic materials. The substrate can comprise any desired form, for example, a card, a plate, a chip, a membrane, a wafer, and other geometries and forms. A substrate can comprise flat surfaces designed to receive or link samples at discrete loci, such as flat surfaces with hydrophobic regions surrounding hydrophilic loci for receiving, containing or binding a sample, wherein a substrate can comprise a planar substrate such as a card or a chip. The substrate can be etched, cut, ground, molded, machined, or otherwise formed, so as to provide one or more fluid processing pathways at least partially defined by the substrate. Exemplary substrate manufacturing techniques that can be used to manufacture the substrate include the techniques described in international patent application publication number WO 2005/029041 to Woudenberg et al., which is incorporated herein in its entirety by reference. The substrate can comprise one or more materials selected from polypropylene, cyclic olefin polymer, or cyclic olefin copolymer, thermoconductive polymers, or fillers, polyethyleneterephthalate, aluminum, gold, iron, copper, zirconium, titanium, alloys of such metals, and the like.

According to some embodiments, one or more fluid processing pathways can each comprise a first region, for example, a pre-amplification region, and can comprise one or more second regions, for example, one or more amplification regions. For example, each fluid processing pathway can comprise two or more second regions, for example, two or more amplification regions. A first region or a pre-amplification region can comprise pre-amplification reaction components. A second region or an amplification region can comprise amplification reaction components. A first region or a pre-amplification region can further comprise sample preparation components, for example, lysis buffer, wherein sample preparation and pre-amplification of nucleic acid contained in the sample, can occur substantially simultaneously in the first region.

According to some embodiments a fluid processing device is provided wherein a fluid processing pathway can further comprise a sample preparation region disposed upstream from and in fluid communication with a first region. The sample preparation region can comprise sample preparation components. The sample preparation region can be in valved fluid communication with the first region, wherein a valve can be disposed therebetween or they can be separated by oil, by a pinch, or by another device feature, or material. Exemplary valves that can be used include those described in U.S. patent application Ser. No. 10/336,274, filed Jan. 3, 2003, and Ser. No. 10/625,449, filed Jul. 23, 2003, both of which are incorporated herein in their entireties by reference. Wax valves can be used.

In some embodiments, a sample preparation region or a pre-amplification region can comprise sample preparation components adapted to separate or isolate nucleic acid sequences from other components of a sample. Sample preparation components can be pre-loaded into a region or can be added to a region by the end-user prior to use. Sample preparation components can comprise, for example, components adapted to lyse cells by suitable methods. Suitable methods can comprise, for example: thermal lysis; mechanical lysis; sonic lysis; chemical lysis; enzymatic lysis; combinations thereof; or the like methods. Sample preparation components can comprise acids, bases, or buffers, that can be adapted to adjust the pH of a fluid sample, and/or membranes, attachment surfaces, beads, or the like.

According to some embodiments a fluid processing device is provided wherein a fluid processing pathway can comprise one or more third regions each disposed downstream from and in fluid communication with a respective, corresponding, second region. The one or more third regions can each comprise, for example, a respective set of sequence reaction components adapted to perform a sequence reaction or real time reaction. One or more amplified target nucleic acid sequences contained in a respective, corresponding, second region. A third region can be in fluid communication with a respective, corresponding, second region. According to various embodiments, a valve can be disposed therebetween or the fluid communication can be valve-less, for example, using oil.

According to some embodiments, a fluid processing device is provided wherein a fluid processing pathway can comprise one or more purification regions disposed downstream from, for example, one or more of a sample preparation region, a pre-amplification region, an amplification region, and a sequencing region, whereby one or more of a pre-amplification product, an amplification product, and a sequencing product, can be purified.

In some embodiments, a fluid processing device is provided wherein a fluid processing pathway can comprise one or more storage regions disposed downstream from and in fluid communication with, a respective, corresponding, second or amplification region, or a respective, corresponding, third or sequencing region, whereby processed fluids can be preserved or stored in the storage region, and can thereafter be accessed or removed from the storage region or from an outlet region disposed downstream of a storage region. For example, processed fluids can be stored in a storage region provided upstream from and in fluid communication with, an outlet region, for example, a dead-end outlet region. The outlet region and a respective storage region can be separated by a valve or oil.

According to some embodiments, one or more second regions of a fluid processing pathway can comprise one or more sealed regions that can comprise, for example, ammonia gas. The one or more sealed regions can comprise pre-loaded ammonia gas. Alternatively the one or more sealed regions can be loaded with ammonia gas by the end-user prior to use. Such loading can be accomplished by, for example, injecting ammonia gas from an ammonia gas cartridge through an access area or port of the one or more sealed regions. The first region can be in fluid communication with and disposed upstream from, one or more sealed regions or from two or more second regions. The one or more fluid processing pathways can comprise at least one valve provided, for example, in fluid communication with and downstream from a first region, and in fluid communication with and upstream from one or more second regions. Two or more second regions can be in fluid communication with each other, for example, the two or more second regions can be serially aligned, or can be in dead-end fluid communication with, for example, a first region.

According to various embodiments, a fluid processing pathway can comprise a first region in valved fluid communication with one or more second regions, for example, one or more sealed regions. The one or more sealed regions can comprise ammonia gas. A fluid processing pathway can comprise a first region in valved fluid communication with two or more second regions, wherein the fluid processing pathway can comprise a valve disposed between and in fluid communication with the first region and the two or more second regions.

In some embodiments, a fluid processing device can comprise a cover layer provided over at least a portion of a top or a first surface of the device to seal one or more exposed elements of one or more fluid processing pathways provided in communication with at least a portion of the top or first surface of the substrate, for example, a cover layer can be provided over openings corresponding to one or more second regions to thereby form one or more sealed regions. The one or more sealed regions can comprise ammonia gas.

According to various embodiments, a fluid processing device can comprise a cover that can be provided on at least a portion of a top surface of a substrate. For example, a cover layer can partially cover one or more of a region, a channel, a duct, and the like. The cover can comprise a removable strip portion that can be provided over, for example, one or more exposed regions. The cover can comprise one or more cover portions. A cover can comprise one or more of a permanently provided cover portion, a semi-permanently provided cover portion, a removably provided cover portion, a re-sealable cover portion, and any combination thereof, by one or more of adhesive sealing, heat sealing, laminating, surface modification, chemical bonding, static forces, and the like. Exemplary card-type device sealing features and systems that can be used include those described, for example, in U.S. patent application Ser. Nos. 11/086,276, 11/086,263, and 11/086,264, all filed Mar. 22, 2005, which are incorporated herein in their entireties by reference.

According to some embodiments, the cover can comprise a flexible material, a rigid material, an elastically deformable material, or a combination of two or more thereof. The cover can comprise a transparent, translucent or opaque material. The cover can comprise an adhesive, flexible sheet. The cover can be provided on at least a portion of a top surface under conditions sufficient to form a liquid-tight seal. The cover can be provided on at least a portion of a top surface under conditions sufficient to form a seal, for example, a gas-tight seal. Liquid-tight seals can be used and can comprise, for example, porous sealing films, layers, or covers, or non-porous, gas-permeable films, layers, or covers, for example, as described in U.S. patent application Ser. No. 10/762,786, filed Jan. 22, 2004, which is incorporated herein in its entirety by reference. The seal can comprise, for example, a single-layer or a multi-layer construction.

Cover layers can comprise, for example, those described in U.S. patent application Ser. No. 10/762,786, filed Jan. 22, 2004, and in U.S. Patent Application Publication No. US 2003/0021734 A1, to VANN et al., filed Aug. 2, 2002, which are incorporated herein in their entireties by reference.

An elastically deformable cover layer can comprise PCR tape materials. Polyolefinic films, other polymeric films, copolymeric films, and combinations thereof can be used, for example, for an elastically deformable cover layer.

According to some embodiments, the microfluidic device can comprise an adhesive layer provided, for example, between a top surface of a substrate and a lower surface of a cover, over a top surface of a substrate, over a lower surface of a cover, or any combination thereof. The adhesive can comprise an adhesive gasket layer provided between the substrate and the cover. The adhesive can comprise any suitable conventional adhesive. For example, an adhesive can comprise one or more of a permanent adhesive, a pressure-sensitive adhesive, a thermo-sensitive adhesive, and a non-permanent adhesive. Silicone pressure sensitive adhesives, fluorosilicone pressure sensitive adhesives, and other polymeric pressure sensitive adhesives can be used. The adhesive can be provided over an entire surface or can be provided over at least a portion of a surface of, for example, a top surface of a substrate or a lower surface of a cover.

According to some embodiments, a first region can comprise pre-amplification reaction components disposed therein. The pre-amplification reaction components can comprise components adapted to pre-amplify a plurality of different nucleic acid sequences present in a sample, for example, a biological sample. Pre-amplification reaction components can comprise any component, reagent, reactant, buffer, marker, primer, probe, label, zip code oligonucleotide, immobilized zip code oligonucleotide, enzyme, nuclease, catalyst, and any other moiety, whose presence is necessary or desired for carrying out a pre-amplification reaction or for carrying out a subsequent reaction to be performed downstream of a first region. A zip-coded oligonucleotide can comprise a sequence, for example, an immobilized sequence, having substantially no homology to a target sequence, as well as, for example, a zip-coded primer sequence having a portion homologous to the zip-coded sequence and a portion homologous to the target sequence. As discussed above, the use of zip code reactants and universal PCR can be used, for example, for a hybridization assay or for real-time PCR.

According to some embodiments, a fluid processing device is provided that can comprise: a substrate that can comprise a first surface and an opposing second surface; and one or more fluid processing pathways that can be at least partially defined by the substrate, the one or more fluid processing pathways each can comprise a first region that can comprise pre-amplification reaction components disposed therein and adapted to pre-amplify a plurality of different nucleic acid sequences present in a sample, to produce a plurality of pre-amplified sequences, and two or more second regions each in fluid communication with the first region and each can comprise amplification reaction components disposed therein adapted to amplify one or more of the plurality of pre-amplified sequences to produce one or more amplified target sequences. In some embodiments the two or more second regions can each be in dead-end fluid communication with the first reaction region. According to some embodiments, the second regions can be vented, for example, with ports, vents, a permeable layer, a porous layer, hydrophobic stops, combinations thereof, and the like. In some embodiments the two or more second regions can each be in fluid communication with the first reaction region and with each other. In some embodiments the fluid processing device can further comprise a cover provided over at least a portion of the top or first surface, that can comprise one or more access areas, wherein an access area can correspond to a region, for example, a first region, to form one or more accessible first regions.

According to some embodiments, a fluid processing device is provided wherein the one or more fluid processing pathways can comprise a channel fluidly connecting a first region and two or more second regions. In some embodiments the channel can comprise at least one valve disposed between the first region and the two or more second regions, and each of the first region and the two or more second regions can be in fluid communication with the valve. The valve can comprise a valve that opens or a valve that opens and closes. According to some embodiments, the at least one valve can comprise a heat-mediated, pressure-actuated valve. The heat-mediated, pressure-actuated valve can comprise a burstable valve. According to various embodiments, wax valves can be used. According to various embodiments, deformable valves can be used. Exemplary valves that can be used include those described in U.S. patent applications Ser. No. 10/336,274, filed Jan. 3, 2003, and Ser. No. 10/625,449, filed Jul. 23, 2003, which are incorporated herein in their entireties by reference.

According to some embodiments, a fluid processing device is provided wherein amplification reaction components can comprise one or more components adapted to amplify at least portions of two or more different sequences of a plurality of pre-amplified sequences. In some embodiments the amplification reaction components can comprise amplification reaction components adapted to amplify three or more different sequences of a plurality of pre-amplified sequences.

In some embodiments, a fluid processing device is provided wherein a first or pre-amplification region is pre-loaded with one or more pre-amplification reaction components.

According to some embodiments, a fluid processing device is provided wherein two or more second or amplification regions can comprise one or more sealed regions, for example, two or more sealed regions. In some embodiments, the one or more sealed regions can comprise ammonia gas. The one or more sealed regions can comprise pre-loaded ammonia gas or ammonia gas can be loaded by the end-user prior to use. In some embodiments the fluid processing device can comprise a cover provided over at least a portion of the top or first surface of the device, that can comprise one or more access areas, wherein an access area can correspond to a region, for example, one or more of a first region and a second region, to form one or more accessible regions.

According to some embodiments, a fluid processing device is provided wherein a first region can comprise one or more buffering components present in an amount sufficient to at least partially adjust and/or neutralize a pH of a fluid sample after a fluid sample is mixed with ammonia gas. The one or more buffering components can comprise one or more acidic, alkaline, or neutral, buffering components.

According to some embodiments, a fluid processing device is provided wherein a first region can comprise one or more sample preparation components. Sample preparation components can comprise, for example, any components necessary or desired that can render one or more nucleic acid sequences present in a fluid sample, available for participation in a process. A process can comprise, for example, a pre-amplification reaction, a purification process, an amplification reaction, a sequencing reaction, or any combination thereof. In some embodiments, sample preparation components can comprise one or more of a lysis buffer, a buffer, an enzyme, ethanol, another alcohol, a precipitating agent, a sequestering agent, or the like.

According to some embodiments, a fluid processing device is provided wherein two or more sealed regions can comprise one or more buffering components sufficient to neutralize a pH of a fluid sample having ammonia gas dissolved therein.

In some embodiments, a fluid processing device is provided wherein each of two or more second regions can comprise a respective set of amplification components adapted to amplify one or more different pre-amplified sequences in each respective second region, and each respective set of amplification components can differ from at least one other set of the respective sets of amplification components. In some embodiments, two or more second regions can comprise three or more second regions.

According to some embodiments, a fluid processing device is provided wherein a first reaction region can comprise one or more sets of pre-loaded immobilized zip-coded oligonucleotides, and two or more second regions can each comprise one or more sets of pre-loaded complementary zip-coded oligonucleotides. Various methods using universal PCR and/or zip codes, that can be carried out according to various embodiments, can comprise the methods and use of the components described, for example, in U.S. patent application Ser. No. 11/090,830 to Andersen et al., and Ser. No. 11/090,468 to Lao et al., both filed Mar. 24, 2005, in U.S. Pat. No. 6,605,451 to Marmaro et al., and in international patent application publication no. WO 2004/051218 to Andersen et al., which are incorporated herein in their entireties by reference. Real time PCR and resequencing are two exemplary methods that can be carried out using such methods and devices.

According to some embodiments, a fluid processing system is provided that can comprise: a fluid processing device; and a detector that can be capable of optical communication with two or more second regions of a fluid processing pathway of the fluid processing device. The detector can be adapted to detect, in the two or more second regions, one or more amplified target sequences that can each be labeled with a respective detectable label. A detectable label can comprise a fluorescent label.

According to some embodiments, a detector can comprise, for example, an LED excitation source and a photodiode detector arranged to excite and detect, respectively, fluorescent dyes. Excitation sources and detectors can comprise those described in U.S. patent application Ser. Nos. 10/205,028, 10/887,486, and 10/887,528, all of which are incorporated herein, in their entireties, by reference. A detector can comprise a spectrophotometer, a fluorometer, an excitation beam source, a charge-coupled device, a camera, or a combination thereof. The detector can comprise, for example, the Applied Biosystems 7500 fast real-time PCR system for providing rapid detection of a broad range of fluorophores, available from Applied Biosystems Corporation, Foster City, Calif.

In some embodiments, a fluid processing system is provided that can comprise a fluid processing device and a thermal cycling device. The fluid processing system can comprise a detector.

According to various embodiments, a fluid processing device is provided that can comprise: a substrate having a first surface and an opposing second surface; and one or more fluid processing pathways that can be at least partially defined by the substrate, the one or more fluid processing pathways can each comprise at least one heat-mediated, pressure-actuated valve adapted to burst when a pressure, for example, that can be at least two atmospheres is exerted across the valve and the valve can be heated to a temperature, for example, of from about 100° C. to about 150° C., of from about 105° C. to 130° C., of from about 110° C. to about 125° C., or greater than about 115° C. Creating pressure to burst the valve can comprise heating the sample at a temperature of grater than 100° C. for a time period of from about one second to about three minutes, for example, from about 10 seconds to about one minute, and/or causing a pressure differential across the valve of from about 0.01 psi to about 100 psi, for example, from about one psi to about 10 psi. The valve can comprise a polymeric, elastomeric, rubber, silicone, and/or plastic material, for example, in the form of a thin layer having an appropriate burst strength and/or tensile strength. The valve can comprise a membrane or plug made of NYLON, TEFLON, aluminum oxide, polyacrylamide, polyethyleneterephthalate, parylene, polystyrene, aluminum, gold, iron, copper, zirconium, titanium, alloys of such metals, and the like. The valve can be circular and have a diameter of from about 0.01 mm to about 10 mm, for example, from about 0.1 mm to about 1 mm. The valve can have a thickness of from about one to about 1000 microns, for example, from about one to about 500 microns or from about 10 to about 100 microns. The valve can comprise, for example, a film of polydimethylsiloxane material that is from about 0.01 to about three millimeters thick. In some embodiments, each of the one or more fluid processing pathways can comprise a first region disposed upstream from and in fluid communication with the at least one heat-mediated, pressure-actuated valve. In some embodiments, the one or more fluid processing pathways can comprise one or more second regions disposed downstream from and in fluid communication with the at least one heat-mediated, pressure-actuated valve. In some embodiments, the one or more second regions can comprise one or more sealed regions that can comprise ammonia gas. The ammonia gas can be pre-loaded into the one or more sealed regions or the one or more sealed regions can be loaded with ammonia gas immediately prior to use by the end-user, for example, by injecting ammonia gas from an ammonia gas cartridge into an access area or port of a sealed region. In some embodiments, an access area or port can comprise a membrane, an adhesive cover, an adhesive tape, a flexible re-sealable cover or tape, a septum, or the like.

According to various embodiments, a fluid processing device is provided that can comprise: a substrate having a first surface and an opposing second surface; and one or more fluid processing pathways that can be at least partially defined by the substrate, the one or more fluid processing pathways can each comprise a first region, and one or more sealed regions disposed downstream from and in fluid communication with the first region, the one or more sealed regions can comprise ammonia gas. In some embodiments, the one or more sealed regions can comprise pre-loaded ammonia gas. In some embodiments, the one of more fluid processing pathways can further comprise at least one channel fluidly connecting the first region and the one or more sealed regions. In some embodiments, the channel can comprise at least one valve disposed between the first region and the one or more sealed regions, and each of the first region and the one or more sealed regions can be in fluid communication with the valve. The valve can comprise a valve that opens or a valve that opens and closes. In some embodiments, a first region can comprise pre-amplification components disposed therein adapted to pre-amplify a plurality of different nucleic acid sequences present in a fluid sample, whereby upon pre-amplifying the fluid sample a plurality of pre-amplified sequences are produced. A first region can further comprise one or more buffering components. A first region can comprise sample preparation components. The one or more buffering components can comprise at least an acidic buffering component. The one or more sealed regions can comprise one or more buffering components sufficient to at least partially neutralize a pH of a fluid upon contact of the fluid with the ammonia gas. In some embodiments, a fluid processing device is provided wherein one or more sealed regions can comprise a plurality of sealed regions, and each of the plurality of sealed regions can comprise a respective set of amplification components adapted to amplify one or more different target nucleic acid sequences of the plurality of pre-amplified sequences. According to some embodiments, each respective set of amplification components can differ from at least one other set of the respective sets of amplification components. In some embodiments, the at least one valve can comprise a heat-mediated, pressure-actuated valve. The heat-mediated, pressure-actuated valve can comprise a burstable valve that can be adapted to burst at a pressure differential across the valve that can be, for example, greater than or equal to about two atmospheres, when the burstable valve can be heated to a temperature, for example, of from about 100° C. to about 130° C., of from about 105° C. to 125° C., of from about 110° C. to about 125° C., or greater than about 115° C. In some embodiments, the one or more sealed regions can comprise two or more sealed regions or at least three sealed regions. According to some embodiments, each of the at least three sealed regions can comprise a different set of amplification components that can be adapted to amplify at least one different target pre-amplified sequence in each respective region, to produce a total of at least three different amplified target sequences.

According to some embodiments, one or more of the one or more fluid processing pathways can comprise one or more valves. The fluid processing device can comprise a series of regions that can be in fluid communication with adjacent regions or can be capable of fluid communication wherein fluid communication is controlled between adjacent regions using, for example, a valve provided between adjacent regions of a fluid processing pathway. A valve can be disposed between adjacent regions to control fluid flow through a channel, flowpath, or fluid processing pathway.

According to some embodiments, a valve can comprise any material, structure, or configuration, that is capable of controlling fluid movement through a pathway, channel, region, or area, upon actuation. The valve can comprise a valve that can be opened, or can be opened and closed. The valve can comprise one or more valves that can be actuated by one or more of, for example, pressure, deformation, solubilization, cutting, heat, and force. According to some embodiments, the one or more valves can comprise one or more of an optical valve, a dissolvable valve, a heat-meltable valve, a heat-mediated pressure-actuated valve, a pressure-actuated valve, a mechanical valve, and a deformable valve, for example, an intermediate wall. The deformable valve and devices for actuating such a valve can comprise those disclosed in United States Patent Application Publication No.: 2004/0131502 A1, to COX, et al., filed Mar. 31, 2003, hereby incorporated by reference in its entirety, herein. Other valves that can be used in the microfluidic device can comprise those disclosed in U.S. Pat. No. 6,817,373 B2, to COX, et al., issued Nov. 16, 2004, and U.S. Patent Application Publication No.: 2004/0055956 A1, to HARROLD, Michael, P., filed Jul. 28, 2003, each of which are hereby incorporated herein in their entirety. Loading can be performed using capillary action, centrifugation, vacuum, pressure differential, or other methods and/or conditions that will be recognizable to those of skill in the art.

In some embodiments, the one or more fluid processing pathways can be provided substantially parallel to each other. A fluid processing pathway can comprise, for example, one or more of a region, an area, an access area, a channel, a branch, and a valve. A region can comprise any shape or form capable of retaining a volume of fluid. For example, a region can comprise a surface area, an area, a recess, a chamber, a depression, a well, a space, or the like. A region can comprise any shape, for example, round, teardrop, square, irregular, ovoid, rectangular, or the like. A region or channel can comprise any cross-section configuration, for example, square, round, ovoid, irregular, trapezoid, or the like. For example, a channel can comprise a cross-sectional area that has an aspect ratio, that is, a width/depth ratio, of greater than one. A channel can comprise a semi-oval cross-sectional area in a substrate. The cross-sectional area can comprise an aspect ratio, that is, a width/depth ratio, of greater than one. A channel can comprise a thin and narrow channel formed in a substrate, wherein the cross-sectional area has an aspect ratio, that is, a width/depth ratio, of less than one. A channel can comprise a trapezoidal cross-sectional area and generally can comprise an aspect ratio of less than one. These and other cross-sectional designs can be used as channels, for example, flow-restricting channels, and can be preformed or formed during a valve-opening operation according to some embodiments.

According to some embodiments, access areas or ports can be provided through for example, one or more of a top or first surface of the fluid processing device, through a bottom or second surface of the device, through a side edge or end edge of the device, through the substrate, through the cover layer, and through a combination of these features. For example, the device can comprise an inlet access area through a cover layer and in communication with an inlet or first region of the device. The device can comprise an outlet access area through a cover layer and in communication with an outlet region.

In some embodiments, a fluid processing device is provided that can comprise one or more fluid processing pathways. A fluid processing pathway can comprise a flow that splits a flowpath into two or more branch channels. The two or more branch channels can comprise two or more substantially parallel branch channels. A first branch channel can comprise, for example, a first amplification region, and a second branch channel can comprise a second amplification region.

According to some embodiments, one or more flow splitters for splitting the fluid sample from one sample into two or more samples or aliquots along two or more branch channels of a fluid processing pathway, can be provided in one or more of the one or more fluid processing pathways, for example, for splitting a sample into 2, 3, 6, 12, 24, 48, 96, 192, or 384 samples or aliquots. According to some embodiments, a flow splitter can be disposed downstream of a first or pre-amplification region, to split the pathway into two or more branch channels or flowpaths. Each branch channel can end at a respective region that can be dead-end or can be open-ended.

Branch channels can be used to obtain equal volumes of fluids in as many portions or aliquots as desired. Branch channels can be in fluid communication with a region, for example a processing region forming individual pathways for further processing of each aliquot. The pathways can be used to perform a single reaction or process, for example, forward sequencing, or can perform multiple same or multiple distinct reactions or processes, for example, PCR, on an aliquot. Components needed to perform a certain reaction or process in a processing region of a pathway, can be pre-loaded in the respective region at the time of manufacture of the microfluidic device, or can be loaded at the time of use.

Fluid processing pathways that can be used in the fluid processing device can comprise those disclosed in U.S. Patent Application Publication No.: 2004/0018116 A1, to DESMOND, et al., filed Jan. 3, 2003, hereby incorporated by reference herein, in its entirety.

According to some embodiments, a fluid processing device is provided wherein incorporation of a pre-amplification zone or well into a card prior to distribution into a plurality of one or more secondary wells. The pre-amplification zone can be loaded or pre-loaded with cDNA or gDNA, according to various embodiments. The amplification can comprise, for example, either PCR or OLA. The pre-amplification well could be sized depending on input sample size and the sensitivity needed. The amount of input material can be much smaller than that needed for low copy expression analysis, or for bacterial detection by SNP analysis using multiple individual reactions. The primers in the plurality of small wells can comprise target specific primers or can comprise zip coded primers, permitting utilization of a common card. If target specific primers are used in the card, the pre-amplification zone can comprise the complete pool of primers, needed for the multiplex reaction, pre-loaded as well. Alternatively, the primers can be loaded with a sample and mastermix.

### III. Methods

According to various embodiments, a method is provided that can comprise: providing a fluid processing device that can comprise one or more fluid processing pathways, each fluid processing pathway can comprise a first region in fluid communication with two or more second regions; introducing a fluid sample that can comprise a plurality of different nucleic acid sequences, into the first region of the fluid processing device; pre-amplifying two or more of the plurality of different nucleic acid sequences in the first region to produce a pre-amplified fluid sample that can comprise two or more different pre-amplified nucleic acid sequences; moving the pre-amplified fluid sample from the first region to the two or more second regions; and amplifying at least one respective target sequence of the two or more different pre-amplified nucleic acid sequences in each of the two or more second regions, to produce at least one respective amplified target sequence in each of the two or more second regions. In some embodiments, moving can comprise moving the pre-amplified fluid sample from the first region, through at least one channel, and into the two or more second regions.

According to some embodiments, a method is provided that can comprise preparing a fluid sample prior to or simultaneous with, pre-amplifying. The step of preparing can comprise lysing cells contained in a fluid sample.

According to some embodiments, a method is provided that can comprise reacting a target nucleic acid sequence to form a detectable label. Labeling can comprise reacting two or more different target nucleic acid sequences and/or probes, each with a different fluorescent label such that two or more different amplified target nucleic acid sequences contained in a single second region, can be detected in that single region.

In some embodiments, a method is provided that can comprise sequencing in a respective, corresponding third region, at least one respective amplified target nucleic acid sequence contained in a respective, corresponding, second region. The method can comprise moving an amplified fluid sample containing one or more amplified target nucleic acid sequences from a second region to a respective, corresponding, third region.

In some embodiments a method is provided wherein the moving through the at least one channel can comprise moving the pre-amplified fluid sample through at least one valve. The moving through the at least one valve can comprise actuating the at least one valve. In some embodiments, the at least one valve can comprise a heat-mediated, pressure-actuated valve that can comprise, for example, a burstable valve. According to various embodiments, actuating can comprise heating the pre-amplified fluid sample in the first region to a temperature sufficient to produce a pressure that, at the temperature, can be sufficient to burst the burstable valve. Heating can comprise heating the pre-amplified liquid sample in the first region to a temperature, for example, of from about 100° C. to about 130° C., of from about 105° C. to 125° C., of from about 110° C. to about 125° C., or greater than about 115° C. In some embodiments, the pressure can comprise a pressure greater than or equal to about 1.5 atmospheres, two atmospheres, three atmospheres, five atmospheres, or higher.

According to some embodiments, a method is provided wherein moving can comprise one or more of moving the pre-amplified fluid sample by capillary action, centripetal force, pneumatic force, hydraulic force, centrifugal force, inducing a positive-pressure mediated flow of the pre-amplified fluid sample, and inducing a negative-pressure mediated flow of the pre-amplified fluid sample. Moving by inducing a negative-pressure mediated flow can comprise inducing a vacuum to draw the pre-amplified fluid sample from the first region. In some embodiments, the two or more second regions can comprise one or more sealed regions that can comprise ammonia gas. In some embodiments, inducing a vacuum can comprise contacting ammonia gas with the pre-amplified fluid sample, wherein a vacuum can be induced as the ammonia gas dissolves into the pre-amplified fluid sample.

In some embodiments, a method is provided wherein pre-amplifying can comprise linearly or exponentially pre-amplifying a plurality of different nucleic acid sequences. The amplifying can comprise exponentially amplifying at least one target nucleic acid sequence of the plurality of different pre-amplified nucleic acid sequences. According to various embodiments, the pre-amplifying and/or amplifying of a nucleic acid can comprise a thermal cycling nucleic acid sequence amplification process or an isothermal nucleic acid sequence amplification process. If a thermal cycling nucleic acid sequence amplification process is used, the process can comprise, for example, a polymerase chain reaction (PCR). The nucleic acid sequence amplification reaction can comprise an exponential amplification process, for example, PCR, or a linear amplification process, as can occur during, for example, Sanger cycle sequencing.

According to various embodiments, a method is provided wherein pre-amplifying can comprise thermal cycling the fluid sample in the first region. Pre-amplifying thermal cycling can comprise at least five thermal cycles. In some embodiments, a method is provided wherein pre-amplifying can comprise one or more reactions selected from a thermal reaction and an isothermal reaction. Pre-amplifying can comprise one or more of a ligase chain reaction, a polymerase chain reaction, a cycle-sequencing reaction, and an OLA reaction.

According to some embodiments, a method is provided wherein amplifying can comprise thermal cycling the pre-amplified fluid sample retained in two or more second regions. The amplifying thermal cycling can comprise from about ten to about forty thermal cycles. Amplifying can comprise one or more exponential amplification reactions. The pre-amplified fluid sample can comprise a buffer component.

According to some embodiments, a method is provided that can comprise a multiplex amplification process, for example, a multiplex PCR process. The process can comprise pre-amplifying a large region encompassing more than one segment of a nucleic acid molecule using primers outside the target area, that can be followed by amplification of each target area using specific primers for each site. The multiplex PCR process can be adapted to minimize some of the limitations of multiplex PCR, for example, partial binding between PCR primers or between PCR primers and other primers or other regions of the genomic DNA apart from the target site, by adjusting thermal cycling conditions as well as the number of thermal cycles performed, thereby minimizing side products and reduced yields of the desired PCR products. Such adaptations and adjustments of the multiplex PCR process can be readily accomplished by one of ordinary skill in the art to which the invention pertains, without undue experimentation. Multiplex amplifications that can be carried out according to various embodiments include those described, for example, in U.S. patent application Ser. No. 11/090,830 to Andersen et al., and Ser. No. 11/090,468 to Lao et al., both filed Mar. 24, 2005, and 60/661,139 filed Mar. 10, 2005, in U.S. Pat. No. 6,605,451 to Marmaro et al., in U.S. Patent Application Publication No. US 2004/0175733, filed Sep. 9, 2004, and in international patent application publication no. WO 2004/051218 to Andersen et al., which are incorporated herein in their entireties by reference.

According to some embodiments, a method is provided that can comprise: providing a fluid processing device that can comprise one or more fluid processing pathways that can comprise a first region, and at least one sealed region disposed downstream from and in fluid communication with the first region, wherein the at least one sealed region can comprise ammonia gas; retaining a fluid sample in the first region; contacting the ammonia gas contained in the at least one sealed region with the fluid sample, wherein the fluid sample can be drawn into the at least one sealed region as the ammonia gas dissolves into the fluid sample.

In some embodiments a method is provided, wherein the at least one sealed region can comprise pre-loaded ammonia gas. The at least one sealed region can be sealed with a gas-impermeable cover that is sufficient to provide a gas-tight seal whereby ammonia gas contained in the at least one sealed region can be stably maintained therein. According to some embodiments a method is provided that can comprise loading ammonia gas into the at least one sealed region. The loading can comprise injecting ammonia gas from, for example, an ammonia gas cartridge into the at least one sealed region through a port or an access area. In some embodiments the port or an access area can comprise a membrane, a septum, a void, an opening, a re-sealable cover, an adhesive cover, a flexible adhesive cover, or any combination thereof. Ammonia gas can be pre-loaded or loaded into at least one sealed region at, for example, ambient pressure, less than ambient pressure, greater than ambient pressure, one atmosphere, less than one atmosphere, greater than one atmosphere, from about ambient pressure to about two atmospheres, or from about ambient pressure to about 1.5 atmospheres. Ammonia gas loaded into at least one sealed region can be maintained in the at least one sealed region at, for example, ambient pressure, less than ambient pressure, greater than ambient pressure, one atmosphere, less than one atmosphere, greater than one atmosphere, from about ambient pressure to about two atmospheres, or from about ambient pressure to about 1.5 atmospheres.

According to some embodiments a method is provided wherein a valve can comprise a heat-mediated, pressure-actuated valve that can comprise, for example, a burstable valve. In some embodiments, opening the valve can comprise heating a fluid sample to a temperature sufficient to produce a pressure that can be sufficient to burst the heat-mediated, pressure-actuated valve. Heating can comprise heating the fluid sample to a temperature, for example, greater than a temperature used for thermal cycling. Heating can comprise heating the fluid sample to a temperature, for example, of from about 100° C. to about 130° C., of from about 105° C. to 125° C., of from about 110° C. to about 125° C., or greater than about 115° C., to produce a pressure that can be, for example, greater than or equal to about two atmospheres, whereby the heat-mediated, pressure-actuated valve can burst.

According to some embodiments, a method is provided wherein a fluid sample can comprise a plurality of different nucleic acid sequences and a first region can comprise pre-amplification components adapted to pre-amplify two or more of the plurality of different nucleic acid sequences. In some embodiments the method can comprise pre-amplifying a plurality of different nucleic acid sequences in a first region to produce a pre-amplified fluid sample comprising one or more target nucleic acid sequences. In some embodiments a method is provided wherein one or more sealed regions can comprise amplification components adapted to amplify one or more target nucleic acid sequences of the plurality of pre-amplified different nucleic acid sequences contained in a pre-amplified fluid sample. The method can comprise forming a fluid communication between the first region and the one or more sealed regions, drawing the pre-amplified fluid sample into the opened sealed regions, and amplifying one or more target nucleic acid sequences in the one or more opened sealed regions, to produce one or more amplified target nucleic acid sequences. In some embodiments, the method can comprise forming a detectable label. The method can comprise detecting the one or more detectable label. The detectable label can comprise a fluorophore and/or fluorescent dye. According to various embodiments, secondary reactions can be carried out in each of the sealed regions and different lengths, sequences, or regions of a DNA sample can be amplified and/or detected in the different respective sealed regions.

According to some embodiments, a method can comprise detecting a product processed in a microfluidic device. Detection can comprise detecting using a system according to some embodiments, or by implementing any of various independent detection systems.

According to some embodiments, a method is provided that can comprise preparing a nucleic acid sample in a sample preparation region, or in a first or pre-amplification region of a fluid processing pathway of a fluid processing device. Preparing a sample can comprise separating, isolating, or extracting nucleic acid sequence-containing components of a cell from other components of the cell by any of a variety of methods. For example, the cell can first be lysed, for example, using sample preparation components that can comprise one or more of enzymes such as e.g. proteinase K or lysozyme, detergents such as SDS, Brij, Triton X 100, Tween 10, and DOC, chemicals such as sodium hydroxide, guanidine hydrochloride, and/or guanidine isothiocyanate, endonucleases, or restriction endonucleases. The resulting cell fragments can be separated from the nucleic acid containing fluid sample. According to various embodiments, lysing can be carried out using mechanical and/or sonic devices, for example, an ultrasonic transducer. The nucleic acid containing fluid sample can then be purified, for example, by chromatography in a region disposed downstream of a sample preparation region. Chromatography can comprise, for example, ion-exchange chromatography column of the type commonly used in the art, or an ion-exchange material or column of the type described in U.S. patent application Ser. No. 10/414,179, filed Apr. 14, 2003, to Lau et al., which is incorporated herein in its entirety by reference. According to various embodiments, purification can comprise filtration through membranes, for example, as describe in U.S. Pat. No. 6,159,368 to Moring et al., which is incorporated herein in its entirety by reference.

According to some embodiments, a method is provided wherein pre-amplification and/or amplification, can comprise one or more of the following methods: a polymerase chain reaction (PCR); a real time (RT) PCR; a ligase chain reaction; an isothermal amplification reaction; or a signal amplification reaction, for example, an Invader® assay (available from Third Wave Technologies, Inc of Madison, Wis.). Although referred to herein as a nucleic acid sequence amplification reaction, it is to be understood that a signal amplification method such as an Invader® assay can be performed instead of actually amplifying or making replicates of a target nucleic acid sequence. More information about the Invader® assay and methods and devices for carrying out such an assay, are described in U.S. Pat. No. 6,706,471 which is incorporated herein in its entirety by reference.

According to various embodiments, a method is provided wherein the nucleic acid sequence amplification and/or pre-amplification can comprise a replication reaction. In some embodiments, methods of amplification and/or pre-amplification can comprise hybridizing one or more nucleic acid templates with smaller complementary “primer” nucleic acids in the presence of a thermostable DNA polymerase and deoxyribonucleoside triphosphates. Upon hybridization of a primer and a template to form a “primed template complex,” DNA polymerase can extend the primer in a template directed manner to yield a primer extension product. Primer extension products can then serve as templates for nucleic acid syntheses. Upon denaturation, the primer extension products can hybridize with primers to form primed template complexes that can serve as DNA polymerase substrates. Cycles of hybridization, primer extension, and denaturation can be repeated many times to exponentially increase the number of primer extension products.

According to some embodiments, a method is provided wherein amplification and/or pre-amplification can comprise thermal cycling. In some embodiments, a fluid processing system is provided that can comprise a fluid processing device and a thermal cycling device. The cycles of hybridization, primer extension, and denaturation can be conducted by cycling the reactants through different temperatures with the thermal cycling device. The specific temperatures used can be based upon the desired base paring efficiency and can be deduced by those skilled in the art, based upon the base composition of the nucleic acid samples and primers.

According to various embodiments, amplification can comprise a real-time PCR (RT PCR) reaction. The RT PCR reaction can be similar to a PCR reaction except that one or more reactant, primer, or other “probe” can be used that is labeled with a marker, for example, a fluorescent dye marker. Any suitable marker, for example, a fluorophore, can be used. Fluorophores can comprise those that can be coupled to organic molecules, particularly proteins and nucleic acids, and that can emit a detectable amount of radiation or light signal in response to excitation by an available excitation source. Suitable markers can include, for example, materials having fluorescent, phosphorescent, and/or other electromagnetic radiation emissions. Irradiation of the markers can cause them to emit light at respective frequencies depending on the type of marker used. Further details of real-time PCR and systems of carrying out real-time PCR can be found, for example, in U.S. Pat. No. 6,814,934 B1 to Higuchi et al, which is incorporated herein in its entirety by reference.

In some embodiments, labeled primers (probes) can further comprise a quenching molecule so that the probe undergoes fluorescence resonance energy transfer (FRET). FRET is a distance-dependent interaction between the electronic excited states of two dye molecules in which excitation is transferred from a donor molecule to an acceptor molecule without emission of a photon. The efficiency of FRET can be dependent on the inverse sixth power of the intermolecular separation, making it useful over distances comparable with the dimensions of biological macromolecules.

FRET type probes or primers can be used with a suitable polymerase. The polymerase can copy a complementary strand of nucleic acid and digest the probes. This digestion can disrupt the FRET and can allow the observance of the reporter dye with equipment know in the art. These observations can be used to track the progress of nucleic acid replication. Other methods that do not use FRET probes and primers, for example, that use intercalating dyes or dark quenchers, can be used instead as will be recognized by those of skill in the art.

According to some embodiments, a method is provided that can comprise amplifying at least one of a plurality of pre-amplified target sequences to form an amplification product. The amplification product can comprise a nucleic acid and the method can comprise subjecting the amplification product to a nucleic acid sequencing reaction. The nucleic acid sequencing reaction can comprise a Sanger cycle sequencing reaction, step-wise sequencing, or a forward/reverse sequencing reaction involving primers.

According to some embodiments, a sequencing method can comprise direct sequencing, step-wise sequencing, Sanger sequencing, cycle sequencing, sequencing by synthesis, fluorescent in situ sequencing (FISSEQ), sequencing by hybridization (SBH), forward/reverse sequencing, pyrosequencing, sequencing using boronated oligonucleotides, electrophoretic, or microelectrophoretic sequencing, capillary electrophoretic sequencing, or other nucleic acid sequencing methods known in the art that can be applied to small sample volumes. Exemplary descriptions of sequencing in various volumes can be found in U.S. Pat. No. 5,846,727 to Soper et al., U.S. Pat. No. 5,405,746 to Uhlen, and Soper et al., Anal. Chem. 70:4036-4043 (1998), all of which are incorporated by reference.

A method for processing a fluid sample can comprise loading a fluid sample into a pre-amplification region loaded with pre-amplification components, pre-amplifying a plurality of different nucleic acid sequences contained in the fluid sample, causing the pre-amplified fluid sample to move to, for example, an amplification region loaded with amplification components, and amplifying one or more target nucleic acid sequences contained in the pre-amplified fluid sample. A valve can be disposed between the pre-amplification region and the amplification region, and can be actuated such that the pre-amplification product can flow to, for example, a pre-amplification purification region that can comprise purification components, for example, purification media. After purification, an optional valve can be actuated and the purified pre-amplified fluid sample can flow through a flow splitter, if provided, and be distributed to a plurality of substantially parallel branch channels. An aliquot of the pre-amplified fluid sample can flow through a respective branch channel and into a respective amplification region that can comprise amplification components adapted to amplify one or more target nucleic acid sequences contained in the pre-amplified fluid sample, where the aliquot is amplified. Amplified product can be detected during and/or after amplification and/or a valve can optionally be actuated and/or a channel can optionally be appropriately configured such that amplification product can flow to, for example, a respective, corresponding, amplification purification region that can comprise purification components, for example, purification media, where the amplified product can be purified. Optionally, the purified amplified product can be detected, or can then flow to, for example, a storage or outlet region. According to various embodiments, purified amplified product can flow to a respective, corresponding, sequencing reaction region that can comprise sequencing components, where the purified amplified product can be sequenced. The sequenced product can then be caused to flow, for example, via one or more of a force, a valve, or an appropriately configured channel, a corresponding sequencing purification region. After purification, the purified sequencing product can be caused to flow to an outlet region or a storage region disposed upstream from the outlet region. The purified sequencing product can be accessed through, for example, a cover layer provided over the outlet region. The fluid sample or fluid product of a process can be caused to flow from one region, channel, or valve, into an adjacent region, channel, or valve, by, for example, centripetal force, capillary action, gravitational force, pneumatic force, pressure, hydraulic force, a negative pressure-mediated flow, a positive pressure-mediated flow, a combination of any two or more thereof, or the like.

### IV. Figures

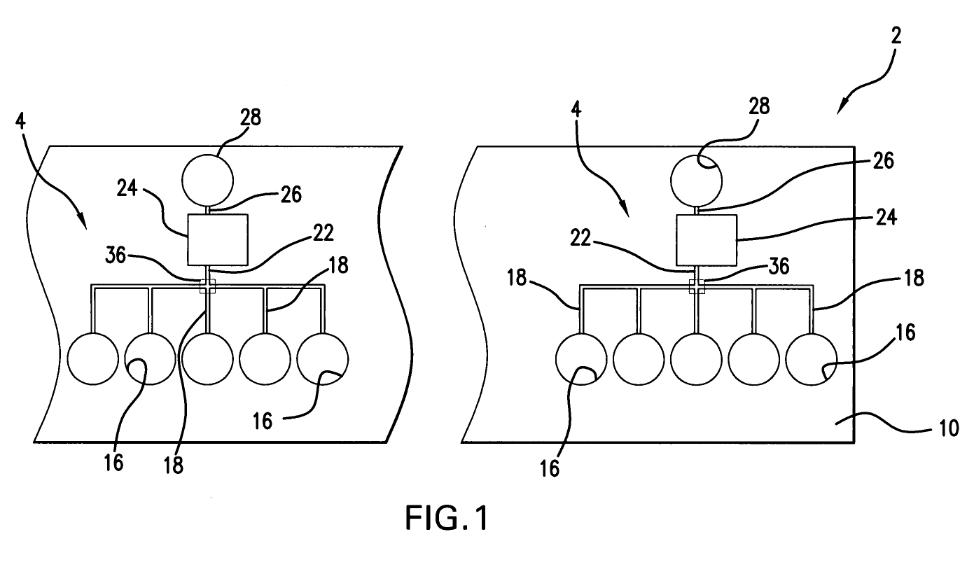

FIG. 1 illustrates an exemplary fluid processing device 2 that can comprise a substrate 10 and one or more fluid processing pathways 4 at least partially defined by the substrate 10. The one or more fluid processing pathways 4 can comprise: a first region 28, for example, a pre-amplification region or a pre-amplification/sample preparation region; a valve 24, for example, a burstable valve; a first channel 26 fluidly connecting the first region 28 and the valve 24; a second channel 22 fluidly connecting the valve 24 to a plurality of branch channels 18, for example, five substantially parallel branch channels 18, wherein the second channel 22 can comprise a plurality of flow splitters 36 that can divert a portion of a fluid sample into each of the branch channels 18; and a plurality of second regions 16, for example, amplification regions, each in fluid communication with a respective branch channel 18. The plurality of second regions 16 can be in dead-end or non-dead-end fluid communication with first region 28. First region 28 can comprise pre-amplification components adapted to pre-amplify a plurality of the same or different nucleic acid sequences present in a fluid sample. First region 28 can further comprise one or more sample preparation components adapted to prepare a sample for pre-amplification such that a plurality of different nucleic acid sequences present in the liquid sample are available for pre-amplification by the pre-amplification components. It is understood that the drawings are schematic and are not drawn to scale. For example, the first region 28 can have a volume equal to the collective volume of all the second regions 16 of the respective fluid processing pathway 4. In the embodiment shown, the volume of the first region 28 can be five times the volume of each second region 16.

Valve 24 shown in FIG. 1 can comprise a valve that is designed only to open or a valve that is designed to open and close. Valve 24 can comprise a heat-mediated, pressure-actuated valve, for example, a burstable valve. Valve 24 can comprise a valve selected from a deformable valve, a dissolvable valve, a meltable valve, an optical valve, a pH sensitive valve, a pressure-actuated valve, and a mechanical valve. A deformable valve, can comprise, for example, an intermediate wall. Each of the flow splitters 36 (three shown in each fluid processing pathway 4) can split a fluid sample into two or more samples or aliquots along two or more branch channels 18 of a fluid processing pathway 4 and can be provided in one or more of one or more section of each fluid processing pathway, for example, to split a sample into 2, 3, 4, 5, 8, 12, 16, 24, 48, 96, 192, 384, 1536, 6144, or more, samples or aliquots. According to some embodiments, and as shown, each flow splitter 36 can be disposed downstream of first region 28. Each branch channel 18 can end at a respective, dead-end, second region 16 or at a respective open-ended second region, for example, at a respective updated reaction site. The fluid processing device 2 can comprise a plurality of fluid processing pathways 4, for example, 2, 4, 8, 16, 24, 48, 96, or 192, or the like, wherein each fluid processing pathway 4 can comprise an independent first region 28 and two or more second or outlet regions 16. Each fluid processing pathway 4 can comprise an independent first region 28 and one or more sealed second regions 16 that can contain ammonia gas.

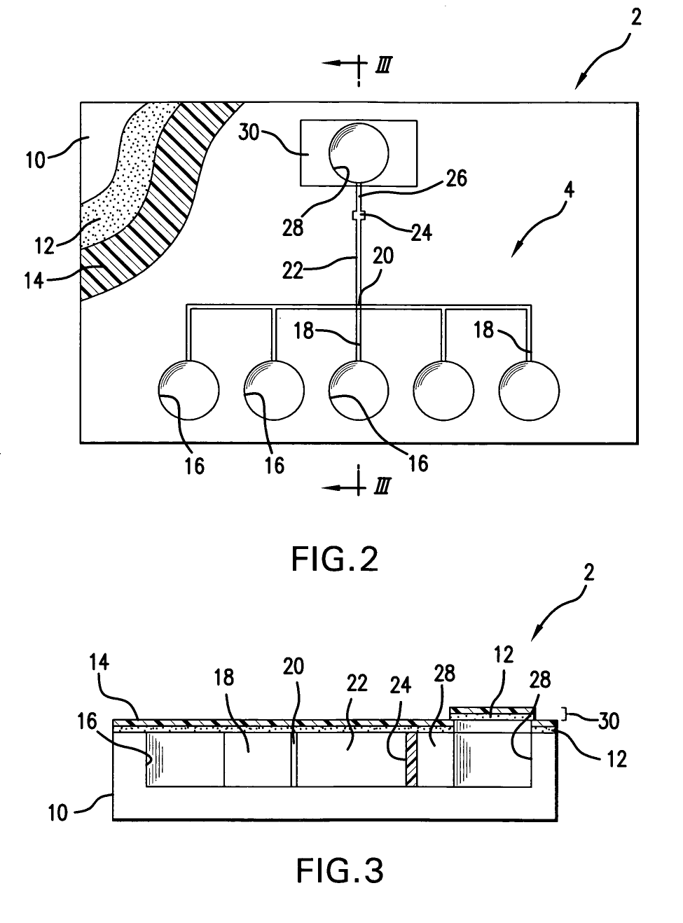

FIG. 2 illustrates a plan view of a fluid processing device 2 that can comprise one or more fluid processing pathways 4, for example, at least partially defined by at least a portion of the substrate 10. The fluid processing device 2 can comprise a cover layer 14 provided over a top or first surface of the substrate 10 and adhered thereto with, for example, an adhesive layer 12. Cover layer 14, can be provided with vents or ports corresponding to each reaction site, or can comprise a gas-permeable cover layer, for example, as described in U.S. patent application Ser. No. 10/762,786, filed Jan. 22, 2004, which is incorporated herein in its entirety by reference. If cover layer 14 is provided ports or vents they can be sealed at an appropriate time, for example, to facilitate loading and prevent evaporation. The fluid processing device 2 can further comprise, before and/or after sample loading, a seal 30, for example, a removable tape, a re-sealable tape, a PCR tape, or a gasket, that facilitates access to the first region 28 of the fluid processing pathway 4. The fluid processing pathway 4 can comprise a first region 28 and a plurality of second regions 16 in fluid communication with first region 28. The fluid processing pathway can comprise at least one channel, for example, a first channel 26 fluidly connecting a first region 28 to a valve 24, for example, a heat-mediated, pressure-actuated valve. Fluid processing pathway 4 can comprise a second channel 22 and a plurality of branch channels 18 fluidly connected to second channel 22, wherein each second region 16 can be fluidly connected to a first region 28 via a respective branch channel 18. The second channel 22 can comprise an intersection 20, for example, a flow splitter as exemplified by reference numeral 36 in FIG. 1.

FIG. 3 illustrates a cross-section view of fluid processing device 2 of FIG. 2 taken through line III-III of FIG. 2. FIG. 3 illustrates a substrate 10 that can comprise a fluid processing pathway provided in communication with, or at least partially defined by, a portion of a top surface or first surface of substrate 10. The fluid processing pathway 4 can comprise a first region 28 in fluid communication with a first channel 26 in fluid communication with a valve 24 that in turn is in fluid communication with a second channel 22 that in turn is in fluid communication with an intersection 20 that in turn is in fluid communication with branch channel 18 that in turn is in fluid communication with a second region 16. Although the first channel 26 and the second channel 22 are shown as having the same depth as the first region 28, the first channel 26 and second channel 22 can each individually instead be deeper or shallower than the first region 28. A flexible cover layer 14 can be provided over at least a portion of a first or top surface of substrate 10 and can comprise and/or be adhered by a corresponding adhesive layer 12. Cover layer 14 can comprise one or more void areas that can, for example, correspond to one or more openings defined by one or more first regions 28. Fluid processing device 2 can further comprise a seal 30 that can comprise, for example, a removable and/or re-sealable tape.

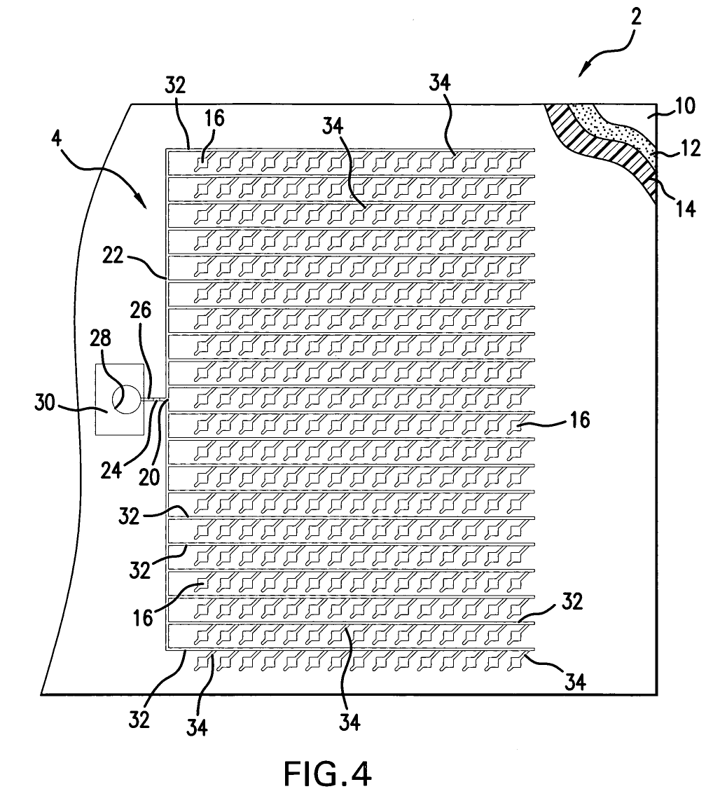

FIG. 4 illustrates a plan view of a fluid processing device 2 that can comprise one or more fluid processing pathways 4 defined by at least a portion of the substrate 10. The fluid processing device 2 can comprise a cover layer 14 provided over a top or first surface of the substrate 10 wherein an adhesive layer 12 can be disposed therebetween to adhere the cover 14 to the top surface. The fluid processing device 2 can further comprise a seal 30, for example, a removable tape, a re-sealable tape, or a PCR tape, that facilitates access to the first region 28 of the fluid pathway 4. The fluid processing pathway 4 can comprise a first region 28 and a plurality of second regions 16 in fluid communication with first region 28. The fluid processing pathway can comprise at least one channel, for example, a first channel 26 fluidly connecting a first region 28 with a valve 24, for example, a heat-mediated, pressure-actuated valve. Fluid processing pathway 4 can comprise a second channel 22 and a plurality of primary branch channels 32 fluidly connected to second channel 22. Each primary branch channel 32 can be fluidly connected to a plurality of secondary branch channels 34, wherein each second region 16 is fluidly connected to a first region 28 via a respective secondary branch channel 34, a primary branch channel 32, a second channel 22, and a first channel 26. The second channel 22 can comprise many intersections 20 that can each comprise, for example, a flow splitter.

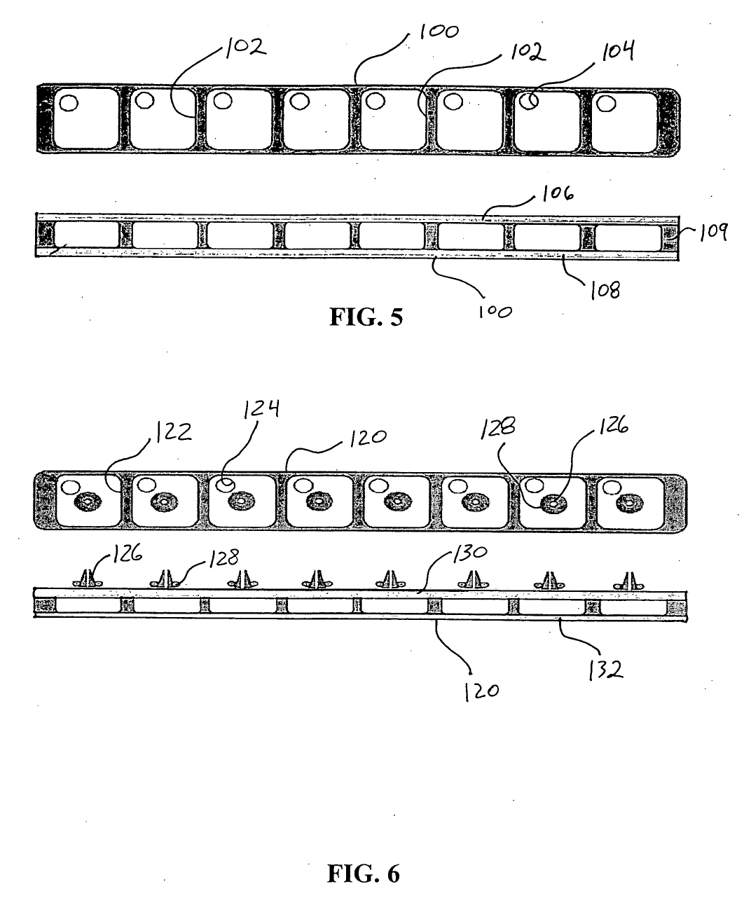

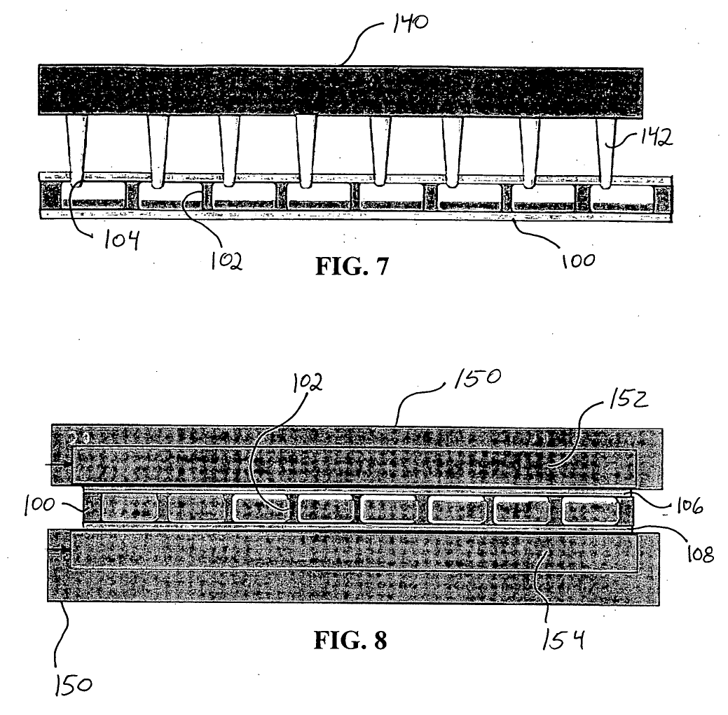

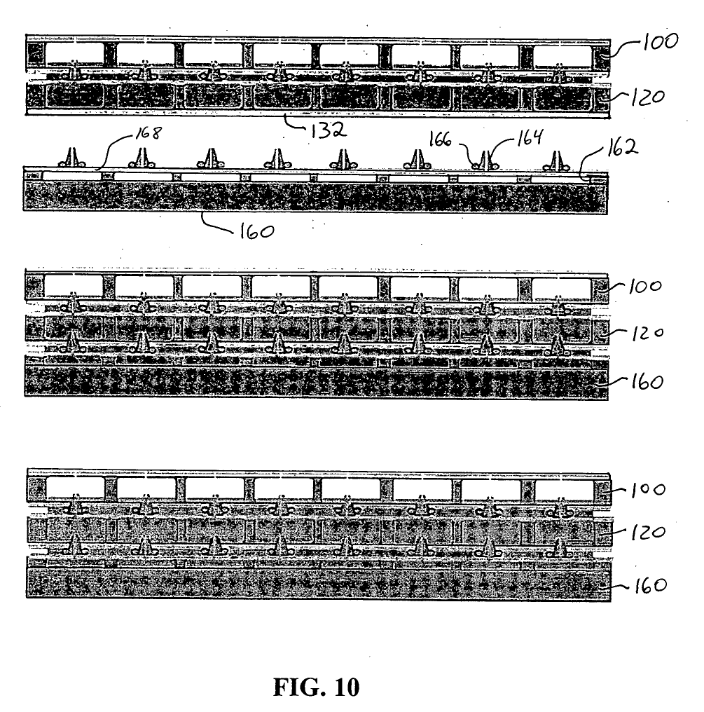

A system according to another embodiment of the present teachings is shown in FIGS. 5-10. The system comprises a pre-amplification array 100 (FIG. 5), a mixing array 120 (FIG. 6), and a microfluidics card 160 (FIG. 10). As shown, for example, in FIG. 5, pre-amplification array 100 comprises a plurality of pre-amplification reaction chambers 102, each provided with an access port 104. To facilitate heating, for example, thermocycling, of contents in reaction chambers 102, pre-amplification array 100 is provided with a thermally conductive top layer 106, a thermally conductive bottom layer 108, and a substrate layer 109 sandwiched between layers 106 and 108. Substrate layer 108 can comprise a polymeric material, for example, poly-carbonate or a poly-cycloolefin copolymeric material. As shown in FIG. 7, pre-amplification array 100 can be filled by a multichannel pipetting device 140, for example, comprising a number of discharge nozzles 142 that corresponds to the number of reaction chambers 102 or a fraction thereof. After loading reaction chambers 102 with reaction components for a pre-amplification reaction, including one or more target sequences to be pre-amplified, pre-amplification array 100 can be sealed, for example, with a PCR tape or other sealing material, to close-off access ports 104. The sealed pre-amplification array 100 can then be thermally cycled, for example, with a thermocycler 150 as shown in FIG. 8. Thermocycler 150 can comprise one or more heating plates although two heating plates 152, 154 are shown in FIG. 8. Thermally conductive layers 106 and 108 can each independently comprise a metal, for example, aluminum or copper, to facilitate heat transfer from thermocycler 150 to the contents of reaction chambers 102.

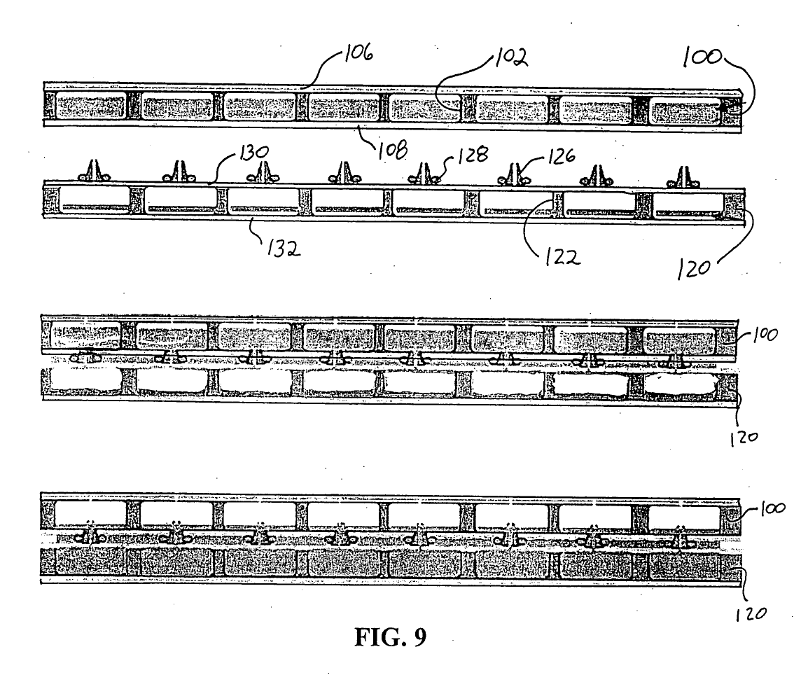

Subsequent to thermally cycling the contents of reaction chambers 102 in pre-amplification array 100, the pre-amplified products from reaction chambers 102 can be transferred into respective reaction chambers 122 of mixing array 120, as shown in FIG. 9. As shown in FIG. 9, mixing array 120 is provided with a plurality of transfer nozzles 126, each having a sharp tip configured to puncture thermally conductive bottom layer 108 of pre-amplification array 100, to form respective fluid communications between reaction chambers 102 and corresponding reaction chambers 122. Mixing array 120 is provided with a top layer 130 and a bottom layer 132 which, in various embodiments, can comprise thermally conductive material, for example, a metal such as aluminum or copper. In the embodiment shown, bottom layer 132 can be configured to be easily punctured as described below in connection with the description of FIG. 10. A plurality of seals can be provided between pre-amplification array 100 and mixing array 120 by a plurality of O-rings 128, one provided around each transfer nozzle 126. A clamp (not shown) can be provided to press pre-amplification array 100 and mixing array 120 together and to maintain sealed fluid communications between reaction chambers 102 and reaction chambers 122. Using centrifugation, the pre-amplified products in reaction chambers 102 can be transferred through transfer nozzles 126 into respective reaction chambers 122. As shown in FIG. 9, reaction chambers 122 can be pre-loaded with reaction components, for example, amplification reaction components, prior to such a transfer process. Pre-loading of reaction chambers 122 can be enabled through access ports 124 (FIG. 6) which can then be subsequently sealed, for example, with PCR tape.

After transfer into mixing array 120, the contents of reaction chambers 122 can be thermally treated, for example, thermally cycled, or, alternatively, transferred to a microfluidics card 160 (FIG. 10) without being heat treated. As shown in FIG. 10, microfluidics card 160 can be provided with a plurality of reaction chambers 162, a plurality of transfer nozzles 164, a plurality of O-rings 166, and a thermally conductive top layer 168, in a configuration similar or identical to that shown for mixing array 120. Microfluidics card 160 and mixing array 120 can be clamped together as shown in FIG. 10 such that transfer nozzles 160 puncture thermally conductive bottom layer 132 of mixing array 120 to provide respective fluid communications between reaction chambers 122 and reaction chambers 162. Using centrifugal force, the contents of reaction chambers 122 can be transferred through transfer nozzles 164 and into reaction chambers 162 for subsequent processing. The subsequent processing can comprise, for example, thermally cycling the contents of reaction chambers 162. In both FIGS. 9 and 10, the top of the drawing shows an arrangement before a clamping operation, the middle of the drawing shows an arrangement after a clamping operation and before centrifugation, and the bottom of the drawing shows an arrangement after clamping and centrifugation.

With the system shown in FIGS. 5-10, a pipette free transfer from a pre-amplification array to a mixing array is provided that reduces or eliminates any risk of cross-contamination between adjacent reaction chambers. Although a linear system of arrays is shown, multi-dimensional arrays can instead be used according to various embodiments.

In the embodiment shown in FIGS. 5-10, exemplary sizes of the various features depicted can include reaction chambers that are 6 mm by 6 mm with a depth of 2.5 mm. The transfer nozzles can extend from about 1.0 mm to about 1.5 mm, from the top surface of the mixing array and/or the microfluidics card.

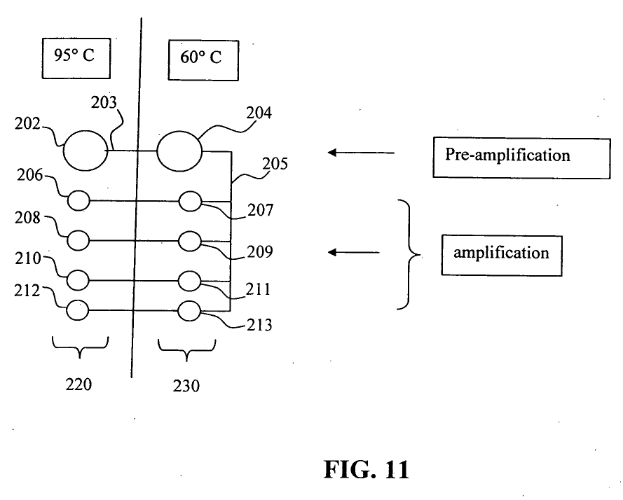

Yet another embodiment of the present teachings is shown in FIG. 11, wherein a system is provided for pre-amplification in a first pair of reaction chambers 202, 204, and amplification is provided in four pairs of reaction chambers 206 and 207, 208 and 209, 210 and 211, and 212 and 213. The system can provide a hot zone 220 and a cool zone 230. A sample can be shifted from hot zone 220 to cool zone 230, and back, to achieve thermal cycling of the sample. For example, thermal cycling to achieve pre-amplification can occur by shifting a sample back and forth between reaction chambers 202 and 204 through a transfer channel 203. Shifting the sample can be provided, for example, according to the teachings of U.S. Pat. No. 5,270,183 or U.S. Pat. No. 5,720,923, both of which are incorporated herein in their entireties by reference. An alternative thermal cycling scheme to provide hot zone 220 and cool zone 230 can comprise shifting the location of a hot plate and/or a cool plate underneath the reaction chambers as described, for example, in U.S. Pat. No. 5,176,203 which is incorporated herein in its entirety by reference. Valves can be provided to control the shifting of sample from hot zone 220 to cool zone 230, and back, and/or centrifugal force can be used to shift the sample.

### V. Examples

### Example 1

A device including 50 different fluid processing pathways as exemplified in FIGS. 2 and 3 can be provided. A user can load 50 different nasal swab samples each into a respective first region, via sample filling ports 1-50. The ports can be closed, for example, by sealing the first regions, and a start button can then be pressed. The first regions can be pre-loaded with lysis buffer and 20 specific primer pairs, random primers, and enzyme (reverse transcriptase or polymerase), whereupon loading a sample, a sample solution can be generated. Exemplary lysis buffers for real-time PCR from direct lysis for a variety of clinical samples are available from many sources, for example, from microzone, zipgen, biovision, and ambion, for example, at www.microzone.co.uk, www.zipgen.com, www.biovision.com, www.ambion.com (RT-PCR compatible cell lysis buffer).

The thermal cycler can be started. Lysis and pre-amplification can then take place in each of the first regions. PCR has been shown to work directly in many lysis buffers. Pre-amplification can occur in a first region having a volume of, for example, from about 100 microliters to about 500 microliters. Thermal cycling can include maintaining an initial temperature of from about 95° C. for about 10 minutes, and then performing 10 cycles each involving heating to 95° C. for about 15 seconds followed by heating to about 60° C. for about one minute.

The thermal cycler can heat the sample solution up to about 110-120° C. The aqueous sample solution can boil and burst the burst valve. Alternatively, the valve can be directly heated, for example, using a heating element that can be formed, in the device, within or adjacent the valve, whereby heating the bulk of the sample above normal thermal cycling temperatures can be avoided. The resultant pre-amplified sample solution in the first region can then flow through a respective first channel to a flow splitter wherein each pre-amplified sample can be split into five aliquots. Each aliquot can then flow through a respective branch channel and into a respective, corresponding second region or second reaction region. The five second regions corresponding to a single fluid processing pathway can each contain four respective pre-loaded sets of nested primers and corresponding probes specific for four different pathogens, plus enzyme and buffer in a dry formulation.

TaqMan cycles can then be run. Amplification can comprise thermocycling for from about 30 to about 40 cycles each comprising heating to about 95° C. for about 15 seconds following by heating to about 60° C. for about 1 minute. Each of the second regions can have a volume of from about 1 microliter to about 10 microliters. 4× multiplexing can take place in each second region to identify the existence or absence of four pathogens per second region and twenty pathogens per sample (5 second regions). Detection and instrument geometry can involve use of an Applied Biosystems (Foster City, Calif.), HT7900 Real-Time PCR detection apparatus. The fluid processing device can contain a total of 300 regions which can include 50 first regions each of which can correspond to five second regions whereby the fluid processing device can include 250 second regions.

Further suitable fluid processing devices, substrates, covers, microfluidic manufacturing methods, input ports, output chambers, pathways, valves, reagents, flow restrictors, valve actuators, cutting tools, and methods of use are described in: U.S. Patent Application Publication No.: 2004/0131502 A1, to COX et al., filed Mar. 31, 2003; U.S. Patent Application Publication No. 2004/0018116 A1 to DESMOND et al., filed Jan. 3, 2003; U.S. Patent Application Publication No.: 2004/0055956 A1 to HARROLD, Michael P., filed on Jul. 28, 2003; U.S. Patent Application Publication No.: 20030152994 A1, to WOUDENBERG et al., filed on Feb. 24, 2003; U.S. patent application Ser. No. 11/029,968, filed on Jan. 5, 2005; and U.S. Patent Application Publication No.: 2004/0018117 A1, to DESMOND et al., filed Jan. 3, 2003, each of which is hereby incorporated herein in its entirety by reference.

Other embodiments of the present teachings will be apparent to those skilled in the art from consideration of the present specification and practice of the present teachings disclosed herein. It is intended that the present specification and examples be considered as exemplary only with a true scope and spirit of the teachings being indicated by the following claims and equivalents thereof.

What is claimed is:

1. A fluid processing device, comprising:
a substrate comprising a first surface and an opposing second surface; and
one or more fluid processing pathways at least partially defined by the substrate, the one or more fluid processing pathways each comprising
a first region comprising pre-amplification reaction components disposed therein and adapted to pre-amplify a plurality of different nucleic acid sequences present in a sample to produce a plurality of pre-amplified sequences, and
two or more second regions each in fluid communication with the first region and comprising amplification reaction components disposed therein adapted to amplify one or more of the plurality of pre-amplified sequences to produce one or more amplified target sequences.

2. The fluid processing device of claim 1, wherein the two or more second regions are each vented.

3. The fluid processing device of claim 1, wherein the amplification reaction components comprise amplification reaction components adapted to amplify at least portions of two or more different sequences of the plurality of pre-amplified sequences.

4. A fluid processing system, comprising:
the fluid processing device of claim 1; and
a detector capable of optical communication with the two or more second regions of each fluid pathway, the detector being adapted to detect, in the two or more second regions, one or more amplified target sequences each labeled with a respective detectable label.

5. A fluid processing device, comprising:
a substrate having a first surface and an opposing second surface; and
one or more fluid processing pathways at least partially defined by the substrate, the one or more fluid processing pathways each comprising
at least one heat-mediated, pressure-actuated valve adapted to burst when a pressure of at least two atmospheres is exerted across the valve and the valve is heated to a temperature of from about 100° C. to about 130° C.

6. A fluid processing device comprising:
a substrate having a first surface and an opposing second surface; and
one or more fluid processing pathways at least partially defined by the substrate, the one or more fluid processing pathways each comprising
a first region; and
one or more sealed regions disposed downstream from and in fluid communication with the first region, the one or more sealed regions comprising ammonia gas.

7. The fluid processing device of claim 6, wherein the one or more sealed regions comprise pre-loaded ammonia gas.

8. The fluid processing device of claim 7, wherein the first region further comprises one or more buffering components sufficient to at least partially neutralize a pH of a fluid sample after communication of the fluid sample with the ammonia gas.

9. The fluid processing device of claim 7, wherein the first region comprises pre-amplification components disposed therein adapted to pre-amplify a plurality of different nucleic acid sequences present in a fluid sample to produce a plurality of pre-amplified sequences upon pre-amplification of the fluid sample.

10. A method, comprising:
providing a fluid processing device comprising one or more fluid processing pathways, each fluid processing pathway comprising a first region in fluid communication with two or more second regions;
introducing a fluid sample comprising a plurality of different nucleic acid sequences from at least one template, into the first region of the fluid processing device;
pre-amplifying two or more of the plurality of different nucleic acid sequences in the first region to produce a pre-amplified fluid sample comprising a plurality of pre-amplified nucleic acid sequences;
moving the pre-amplified fluid sample from the first region to the two or more second regions; and
amplifying at least one respective target nucleic acid sequence of the plurality of pre-amplified nucleic acid sequences in each of the two or more second regions, to produce an amplified fluid sample comprising at least one respective amplified target nucleic acid sequence in each of the two or more second regions.

11. The method of claim 10, wherein the one or more fluid processing pathways each further comprise at least one channel fluidly connecting the first region and the two or more second regions.

12. The method of claim 11, wherein moving comprises moving the pre-amplified fluid sample from the first region, through at least one channel, and into the two or more second regions.

13. The method of claim 10, wherein moving comprises centrifuging the fluid processing device.

14. The method of claim 10, wherein the amplifying comprises exponential amplification.

15. The method of claim 10, wherein the pre-amplifying comprises thermal cycling the fluid sample in the first reaction region.

16. A method, comprising:
providing a fluid processing device comprising one or more fluid processing pathways each comprising a first region, and at least one sealed region disposed downstream from and in fluid communication with the first region, wherein the at least one sealed region comprises ammonia gas;
retaining a fluid sample in the first region;
contacting the ammonia gas contained in the at least one sealed region with the fluid sample, wherein the fluid sample is drawn into the at least one sealed region as the ammonia gas dissolves into the fluid sample.

17. The method of claim 16, wherein the at least one sealed region comprises pre-loaded ammonia gas.

18. The method of claim 16, further comprising loading ammonia gas into the at least one sealed region.

19. The method of claim 16, wherein the one or more fluid processing pathways further comprises a valve disposed between and in fluid communication with the first region and at least one sealed region.

20. The method of claim 19, wherein the valve comprises a heat-mediated, pressure-actuated valve.
